(19) United States
(12) Patent Application Publication
(10) Pub. No.: US 2026/0162013 A1
(43) Pub. Date: Jun. 11, 2026

(54) SYSTEM AND METHOD FOR SECURE MANAGEMENT, LINKING, OPERATIONS TO GENERATE INSIGHTS AND ACCELERATE ANALYTICS AND AI MODELING

(71) Applicant: Privacy Analytics Inc., Ottawa (CA)

(72) Inventors: Lon Michel Luk Arbuckle, Ottawa (CA); Devyani Priyambada Biswal, Toronto (CA); Muhammad Oneeb Rehman Mian, Ottawa (CA)

(21) Appl. No.: 19/400,739

(22) Filed: Nov. 25, 2025

Related U.S. Application Data
(63) Continuation of application No. 19/377,688, filed on Nov. 3, 2025.
(60) Provisional application No. 63/715,972, filed on Nov. 4, 2024.
(60) Provisional application No. 63/789,739, filed on Apr. 16, 2025.

(51) Int. Cl. G06N 20/00

(52) U.S. Cl. CPC G06N 20/00

(57) ABSTRACT

A method for generating a trained machine learning model trained on multiple segregated data sources includes generating a first dataset by transforming a first source dataset by generating an embedded representation of the first source dataset and adding privacy parameters. The method includes generating a second dataset by transforming a second source dataset by generating an embedded representation of the second source dataset and adding privacy parameters, generating a combined dataset that includes the first dataset and a ground truth dataset from a first segregated data environment combined with the second dataset from a second segregated data environment (e.g., within a trusted research environment or a secure processing environment). The method includes training a machine learning model with training data that includes a subset of the combined dataset, in which the model parameters of the trained machine learning model are stored in a storage device.

### CROSS-REFERENCE TO RELATED APPLICATIONS

This application is a continuation of U.S. patent application Ser. No. 19/377,688, filed Nov. 3, 2025, which claims the benefit of priority under 35 U.S.C. § 119 to U.S. Provisional Patent Application . No. 63/715,972, filed on Nov. 4, 2024, and to U.S. Provisional Patent Application No. 63/789,739, filed on Apr. 16, 2025, the entire contents of all of which are incorporated by reference herein.

### BACKGROUND

This specification relates to analyzing and combining data from multiple data environments.

Data are often segregated across different systems and organizations due to privacy laws, intellectual property concerns, and regulatory requirements. For instance, health data is subject to regulations such as the Health Insurance Portability and Accountability Act (HIPAA), which restricts the sharing of sensitive patient information without appropriate safeguards. Similarly, consumer data, including purchase histories and behavioral information, is governed by regulations such as the General Data Protection Regulation (GDPR) to protect individual privacy and consent. These restrictions provide challenges to combining different types of data in a shared data environment for analytics and machine learning applications, even though such integration could provide valuable insights for a variety of applications.

### SUMMARY

The systems and techniques described here related to storing, processing, combining, and analyzing data stored in segregated data environments.

In some implementations, the data relate to healthcare data, consumer data, and other data associated with individuals. In many cases, particular types of data cannot be directly combined and jointly analyzed or processed due to privacy, security, and other regulations.

The disclosed techniques of this specification allow for combining data from segregated data environments and using the combined data for training machine learning models and performing other analyses while minimizing a risk of disclosure of sensitive information and minimizing risk of violating associated privacy, security, intellectual property, and/or contractual restrictions.

The systems described here are designed to operate in accordance with artificial intelligence (AI) governance protocols, privacy operations, performance monitoring, and secure data management. As such, appropriate steps are described to access sensitive data for training machine learning models and other analytical tasks while minimizing a risk of disclosure. Furthermore, the disclosed techniques include an agentic system for identifying data, applying privacy parameters, storing data, and combining data for processing by a machine learning model training agent. In some implementations, other agents are implemented to perform specific tasks including feature engineering, machine learning model inference, model validation, among others. In some implementations, various agents described in the present disclosure provide data to and receive data from an external system (e.g., an analyst via a user interface) as feedback and/or requests and triggers.

The subject matter described in this specification can be implemented in particular embodiments to realize one or more of the following advantages. Techniques are described for implementing a method of combining data from more than one segregated data environment to be used for training a machine learning model or other analytical tasks. The described techniques include transforming the data such that data from multiple segregated data environments can be combined and jointly analyzed while minimizing risk of disclosing personally identifiable information and minimizing risk of violating various regulations related to data privacy. The transformed data are represented as synthetic trends and are resilient to reverse engineering back to the original source data stored in the segregated data environment, increasing the security of the stored data. The generation of the synthetic trends is described in the disclosure of U.S. Patent Application Publication No. US-2025-0265373-A1, filed on Feb. 14, 2025, and is hereby incorporated by reference in its entirety.

In some embodiments, the segregated data environments are instantiated inside a trusted research environment (TRE) or secure processing environment (SPE), enabling secure, purpose-bound access and auditability while the system combines privacy-enhanced artifacts for analytics and model training. The TRE/SPE represent a governed compute enclave that enforces purpose-limited access, role-based controls, audited execution, rate limiting, and data minimization. The TRE/SPE hosts local feature stores and model serving layers and exposes only protected outputs (e.g., outputs of machine learning models that do not sacrifice privacy and/or confidentiality of underlying data). Examples of protected outputs are synthetic trends, aggregates, model coefficients with valid intervals. In the context of the present specification, the TRE/SPE is an instantiation of the segregated environments from which data are transformed and linked while minimizing a risk of disclosing underlying data records.

Some embodiments of the system described in this specification provide an improved functionality of computing infrastructure by enabling a reduced memory footprint and reduced input and output operation requirements by implementing a compact latent embedding space representation of source datasets. The compact latent embedding space is generated by employing adaptive compression and dimensionality reduction that are calibrated to satisfy privacy and confidentiality thresholds while preserving model-useful signals.

Some embodiments of the system described in this specification enable locality-optimized processing using linkable synthetic trends data, in which data processing and storing are implemented in local feature stores and serving layers, thereby reducing cache misses and page faults during training machine learning models and inference operations. Furthermore, the locally-stored data is accessible to entities outside of the local environment with appropriate user access controls, ensuring accessible and secure data storage.

Some embodiments of the system enables streaming joins over tokenized linking keys such that cross-environment linkage can be executed as a sequence of constant-time lookups with bounded working sets. This decreases contention on persistent storage and mitigates head-of-link blocking in multi-tenant environments.

Some embodiments of the system enable reduced inference latency for trained machine learning models by storing frequently-used data aggregates in a secure feature store and by exploiting vectorized execution paths and hardware acceleration for distance computations on the embedded vector spaces.

Some embodiments of the system provide improved fault tolerance and repeatability of machine learning workloads through idempotent task orchestration with rollback semantics and monotonic lineage recording, such that partial failures avoid full pipeline re-execution.

The described systems include automated agentic systems that operate semi-autonomously while interacting with one or more external resources (e.g., receiving feedback from external inputs via communication with a user interface). The agentic nature of the described system allows for flexible and safe generation of trained machine learning models.

The described methods include a combination of multiple data sources. For example, a first data source can be related to health data and a second data source can be related to consumer data. In some cases, a joint analysis of health data and consumer data as it relates to particular individuals can increase outreach efficiencies for certain business objectives. A tokenization of the consumer data and de-identification of the health data combined with a representation of each in embedded vector spaces results in synthetic trends datasets that can be safely combined and analyzed, providing rich health-consumer insights while minimizing risks of violating information security regulations.

Furthermore, the described systems include automated monitoring procedures to ensure data are available, compliant with regulations and protocols, and outputs adhere to expected performance metrics throughout every stage of a data processing pipeline. In some implementations, the system generates status alerts to a user interface for review and receives feedback to modify and improve the data processing pipeline. As such, the data processing pipeline is responsive to corrective feedback to ensure output data and trained machine learning models are generated according to expected criteria.

The described methods include methods of performing inferential bridging. Inferential bridging provides access to analytics on protected data (e.g., healthcare data related to individuals) without sacrificing confidentiality and privacy of the data principals associated with the protected data. Privacy enhancements are implemented on the protected data itself, rather than the procedures for processing the data. This allows analysts to implement preferred statistical and analytics tools without modification, as the data processed by the tools is confidential and private. The inferential bridge provides a safe workbench for access the protected data. In one implementation, the safe workbench is provided inside a TRE/SPE, where user code executes against protected interfaces to synthetic trends, linkable tokens, or sufficient statistics rather than raw row-level data.

The inferential bridge implements a variety of data processing steps including dimensionality reduction and clustering to focus privacy enhancing techniques (e.g., introduction of noise and randomization) where variance and risk is concentrated. This minimizes an amount of noise that is required to reach particular risk thresholds and results in lower computational load and memory usage compared to uniform noise application. The inferential bridge also balances data sets that have under or over-represented attributes. This allows for streamlined training of machine learning models and supports real-time model adaption which reduces computation time and resource requirements during training. The inferential bridge allows secure access to protected data stored in federated and containerized data stores. The data does not traverse the inferential bridge to an end user, but rather insights derived from the data. This preserves a known degree of confidentiality and privacy while allowing useful insights to be extracted from the data.

In an aspect, a system includes one or more computers and one or more computer-readable media storing instructions that are operable, when executed by the one or more computers, to perform operations for generating a trained machine learning model trained on a plurality of segregated data sources. The operations include generating a combined dataset comprising a first dataset and a ground truth dataset from a first segregated data environment combined with a second dataset from a second segregated data environment, wherein the first dataset and the ground truth dataset are stored in a first bridge database, and the second dataset is stored in a second bridge database, wherein the first dataset is a transformation of a first source dataset, wherein the first dataset is generated by generating an embedded representation of the first source dataset and adding privacy parameters to the first source dataset, and wherein the second dataset is a transformation of a second source dataset, wherein the second dataset is generated by generating an embedded representation of the second source dataset and adding privacy parameters to the second source dataset. The operations include training a machine learning model with training data, the training data comprising a subset of the combined dataset, wherein model parameters of the trained machine learning model are stored in a storage device.

Embodiments can include one or any combination of two or more of the following features.

In some implementations, the first source dataset corresponds to health data. In some implementations, the privacy parameters comprise injected noise. In some implementations, the second source dataset corresponds to consumer data. In some implementations, the transformation of the first source dataset and the transformation of the second source dataset are each performed by processing the respective dataset with an embedding neural network model.

In some implementations, the transformation of the first source dataset is performed by a first artificial intelligence (AI) agent operating within the first segregated data environment, and wherein the transformation of the second source dataset is performed by a second AI agent operating within the second segregated data environment. In some implementations, the second AI agent is configured to receive the transformation of the first source dataset from the first AI agent using a model context protocol (MCP) framework of communication between AI agents.

In some implementations, training the machine learning model is performed by a model training artificial intelligence (AI) agent operating within a model training environment, wherein the model training environment is different from the first segregated data environment and different from the second segregated data environment.

In some implementations, the operations further comprise validating the trained machine learning model by a model validation artificial intelligence (AI) agent, wherein the validation comprises evaluating the trained machine learning model based on a training dataset, and wherein the model validation AI agent is configured to receive model parameters of the trained machine learning model. In some implementations, the validation further comprises verifying calibration of predicted probabilities. In some implementations, the operations further comprise transmitting, from the model validation AI agent to an entity operating within a model serving data environment, results of the validation. In some implementations, the operations further comprise storing the model parameters of the trained machine learning model in a storage device within a model serving data environment. In some implementations, the operations further comprise, receiving, at a model inference AI agent operating within the model serving data environment, a task signal from the entity operating within the model serving data environment, wherein the task signal initiates a model inference process performed by the model inference AI agent. In some implementations, the operations further comprise loading, by the model inference AI agent, the model parameters of the trained machine learning model from the storage device within the model serving environment to perform the model inference process. In some implementations, the operations further comprise transmitting, from the model inference AI agent to a delivery AI agent operating within the model serving data environment, results of the inference process, wherein the delivery AI agent is configured to package the results of the inference process for consumption by a second entity operating within the model serving data environment.

In some implementations, the first dataset and the second dataset each include one or more data elements associated with a shared individual, wherein each data element comprises a linking key that links a data element of the first dataset with a data element of the second dataset. In some implementations, the operations further comprise generating a linking database comprising the first dataset, the second dataset, and corresponding linking keys, wherein each linking key is associated with a particular individual.

In some implementations, the operations further comprise selecting a model training strategy from a strategy library database and training the machine learning model according to the selected model training strategy, wherein the strategy library database comprises a plurality of model training strategies.

In an aspect, combinable with the previous aspect, a method for generating a trained machine learning model trained on a plurality of segregated data sources includes the operations described above.

In an aspect, combinable with one or more of the previous aspects, one or more non-transitory computer readable media storing instructions that, when executed by at least one processor, cause the at least one processor to generate a trained machine learning model trained on a plurality of segregated data sources by performing the operations described above.

In an aspect, combinable with one or more of the previous aspects, a method includes retrieving data from a data store according to a data access mode, wherein the data access mode is determined based on a policy profile associated with a data processing job submitted by a user, determining one or more distributional properties of the data, determining one or more risk metrics based on the distributional properties of the data, determining a strategy for adding noise to the data based on the one or more risk metrics, wherein the strategy comprises an amount of noise to add to the data and an optimization strategy for adding the noise to the data, adding the noise to the data according to the determined strategy to generate noisy data, and executing the data processing job, comprising processing the noisy data according to the data processing job to generate an output.

Embodiments can include one or any combination of two or more of the following features.

In some implementations, the method includes determining one or more distributional properties of the noisy data and evaluating one or more updated risk metrics based on the distributional properties of the noisy data.

In some implementations, the method includes determining the one or more updated risk metrics exceed a risk budget, wherein the risk budget is defined in the policy profile and responsive to determining that the one or more updated risk metrics exceed the risk budget, updating the strategy for adding noise to the data based on the one or more updated risk metrics.

In some implementations, the method includes adding noise to the data according to the updated strategy to generate updated noisy data and executing the data processing job, comprising processing the updated noisy data according to the data processing job to generate an updated output.

In some implementations, the method includes loading the policy profile associated with the data processing job submitted by the user and determining the data access mode based on the data processing job and the policy protocol.

In some implementations, the policy profile defines a risk budget associated with the data processing job.

In some implementations, the data access mode is a synthetic data access mode that comprises delivering synthetic data to the user. In some implementations, the data access mode is a pseudonymized data access mode that comprises providing view-only data access to the user. In some implementations, the data access mode is a federated data access mode that comprises delivering protected insights to the user, wherein the protected insights are derived from the noisy data.

In some implementations, the method includes determining a calibration error of the retrieved data and modifying the retrieved data based on the determined calibration data.

In some implementations, the one or more risk metrics comprise records at risk, attributes at risk, and expected shortfall. In some implementations, the optimization strategy comprises a risk-first strategy. In some implementations, the optimization strategy comprises a utility-first strategy. In some implementations, the optimization strategy comprises a balanced strategy that comprises a risk threshold and a utility threshold.

In some implementations, the method includes performing a record-level balancing of the data, the record-level balancing comprising modifying a number of records from the data associated with a particular classification. In some implementations, the method includes performing an algorithm-level balancing of the data comprising modifying classification weights of a machine learning model, wherein the classification weights are associated with a particular classification of a record.

In some implementations, the method includes performing a principal component analysis of the data to determine a plurality of dimensions that characterize the data, wherein the plurality of dimensions represent a subset of dimensions with the highest variance and adding the noise to the data along the plurality of dimensions.

In some implementations, the method includes logging, in a provenance log, the determined strategy for adding noise.

In an aspect, combinable with one or more of the previous aspects, a system that includes one or more computers and one or more computer-readable media storing instructions that are operable, when executed by the one or more computers, to perform operations associated with the method described above.

In an aspect, combinable with one or more of the previous aspects, one or more non-transitory computer readable media storing instructions that, when executed by at least one processor, cause the at least one processor perform operations associated with the method described above.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1 illustrates an example system for processing synthetic trends data with a trained machine learning model.

FIG. 2 illustrates an example system for performing analytics on data from combined segregated data sources.

FIG. 3A illustrates an example process for generating a trained machine learning model trained on synthetic trends data.

FIG. 3B illustrates a continuation of the example process for generating the trained machine learning model trained on synthetic trends data.

FIG. 4 illustrates an example scenario that includes AI agents for performing processes related to generating a trained machine learning model and performing inferences using the trained machine learning model.

FIG. 5 illustrates an example environment in which a first AI agent and a second AI agent interact under an appropriate secure communication protocol.

FIG. 6 illustrates example databases implemented in various data environments.

FIG. 7 illustrates an example system for executing various processes with AI agents operating within associated data environments.

FIG. 8 is an example system for implementing AI governance and privacy operations related to training a machine learning model with data from segregated data environments.

FIG. 9 illustrates an example analytics and consumption environment for monitoring and serving a trained machine learning model.

FIG. 10 illustrates an example process implemented by a processor to determine a task type.

FIG. 11 illustrates an example segregated data environment for performing data processing tasks including generating synthetic trends data.

FIG. 12 illustrates a segregated data environment with AI agents that perform a respective process.

FIG. 13 illustrates an example linking data environment linking data associated with a particular entity stored in multiple segregated data environments.

FIG. 14 illustrates an example model serving environment and an example model training environment for training the trained machine learning model.

FIG. 15 is a flow diagram of an example process for training a machine learning model on combined segregated data sources.

FIG. 16 illustrates an example system that includes an inferential bridge between an end user and an environment.

FIG. 17 illustrates an example system that incorporates features of a model training environment and an analytics environment.

FIG. 18 is a flow diagram of an example process for implementing inferential bridging.

FIG. 19 illustrates an example process that includes a transformation of information and data assets.

FIG. 20 illustrates an example system that includes a control plane, a user plane, and a data plane.

FIG. 21 illustrates a representation of an example process for determining a data optimization strategy.

FIG. 22 illustrates an example system for implementing functionality of an insight command.

FIG. 23 illustrates an example process that represents functionality of an insight command system.

FIG. 24 illustrates an example system that includes an insight command and an insight sentry.

FIG. 25 illustrates an example system that includes a user interface in a user plane communicatively coupled to a control plane.

FIG. 26 illustrates an example system that includes a user interface in a user plane communicatively coupled to a control plane.

FIG. 27 illustrates an example system that includes a chat interface in a user plane communicatively coupled to a control plane.

Like reference numbers and designations in the various drawings indicate like elements.

### DETAILED DESCRIPTION

The systems and techniques described here relate to methods for generating a trained machine learning model trained on multiple segregated data sources. In some cases, data stored in a first segregated data environment are associated with healthcare data of individuals and data stored in a second segregated data environment are associated with consumer data associated with a subset of the same individuals. Due to various privacy and security regulations (among others), certain applications are prohibited from combining data from the first segregated data environment with data from the second segregated data environment. However, in some cases, applications can benefit from combining data from each segregated data environment without compromising privacy of the individuals and without violating relevant regulations that govern storage and usage of the stored data.

The segregation of data environments (also sometimes known as a federated data environment) creates a flexible and safe environment for building and deploying machine learning models. As described throughout the present specification, the generation of training data for training machine learning models, training and validating the machine learning models, and serving the machine learning models to end users can be facilitate with specialized artificial intelligence (“AI”) agents that are configured to interact with external resources (e.g., analysts) as well as other AI agents operating in a common or a different data environment.

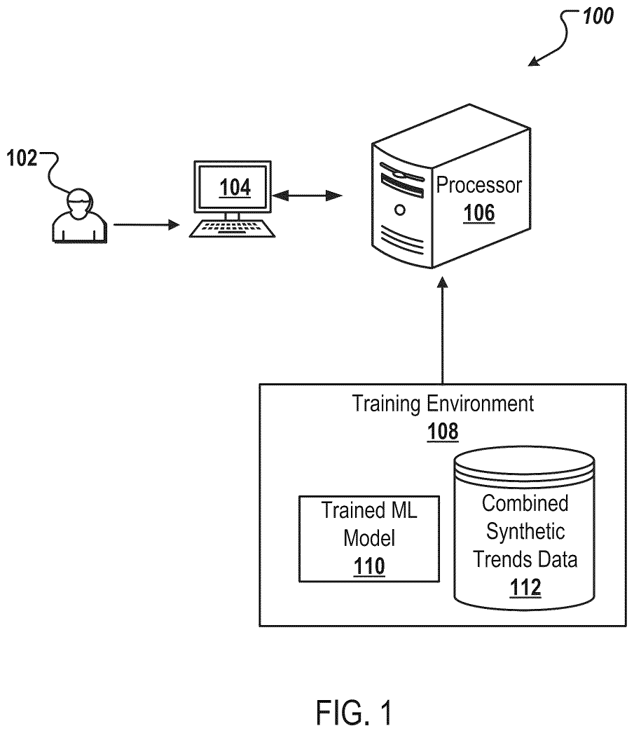

FIG. 1 illustrates an example system 100 that includes a server 106 configured to process synthetic trends data 112 with a trained machine learning model 110. The example system 100 illustrates an example use case of implementing a trained machine learning model using synthetic trends data, in which details of the approach are further described throughout the present specification.

The synthetic trends data 112, which serve as training data and in some implementations, input activation data to the trained machine learning model 110, are synthetic representations of source data (e.g., healthcare data). The synthetic trends data 112 are linked among each individual represented in the source data and can be used for model training and inference without exposing the source data. The synthetic trends data 112 act as a privacy-preserving interface to the source data (e.g., a method for accessing features of the source data without accessing the source data itself). The synthetic trends data 112 are generated using dimensionality reduction, de-identification, and noise injection, producing compact vector representations of the source data that captures useful patterns while minimizing risk of reverse engineering.

The server 106 includes one or more processors that execute instructions associated with the trained machine learning model 110. The system 100 includes a user 102 that interacts with a user device 104 via a user interface. The user device 104 is communicatively coupled to the server 106.

In some implementations, a request for an analysis of data or a request for a machine learning model inference associated with more than one segregated data source (e.g., a healthcare data source and a consumer data source, in which each includes data associated with a subset of shared individuals and entities). The server 106 receives the request from the user device 104 and executes instructions of the trained machine learning model 110.

In some cases, the machine learning model inference can relate to a prediction of a particular action taken by an individual based on consumer activity and health characteristics, e.g., a likelihood of purchasing a particular healthcare product, a likelihood of clicking on a particular advertisement related to a healthcare product, among other possible insights related to consumer activity and health characteristics. For example, an end user can request for a likelihood that a particular person purchases a particular medical device. The system can process consumer data and health data related to the particular person, which are prohibited from being combined in some scenarios due to various regulatory and security restrictions, with a trained machine learning model to generate a data value indicative of the requested likelihood.

The trained machine learning model 110 is trained in a training environment 108 with training data represented as the synthetic trends data 112, and in the case of training data associated with multiple data environments, combined synthetic trends data, stored in a database accessible to a processor of the training environment 108. The synthetic trends data 112 includes data from each of the more than one segregated data sources such that a risk of disclosing private information about the shared individuals and entities is minimized, as described throughout the current specification.

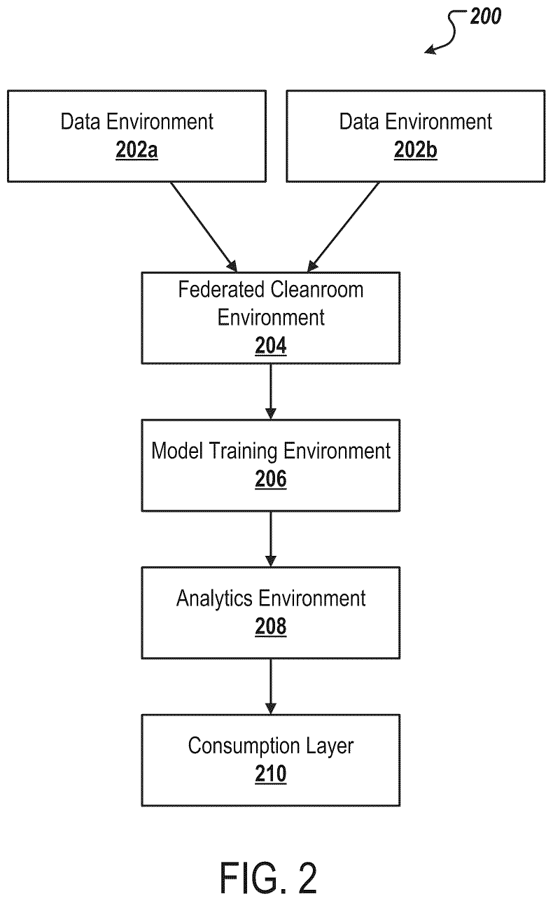

FIG. 2 illustrates an example system 200 for performing analytics on data from combined segregated data sources. The system 200 includes multiple data environments. Each data environment represents a system in which data are collected, stored, processed, and managed. In some cases, each environment is associated with an associated set of controls, rules, and governance protocols.

The system 200 illustrates example relationships between the multiple data environments that interact for combining data from a first data environment 202a with data from a second data environment 202b to generate a trained machine learning model trained on the combined data. In some cases, data stored in the first data environment 202a is prohibited from being directly combined with data stored in the second data environment (e.g., health data and consumer data) due to one or more security, regulation, and privacy protocols.

The system 200 includes a federated cleanroom environment 204 that receives data from the first data environment 202a and the second data environment 202b. The data received from each environment 202a-b are represented as synthetic trends data. For example, the data can be represented as embedded representations of healthcare and/or consumer data, with one or more privacy enhancing techniques applied to the data before being received by the federated cleanroom environment 204. The federated cleanroom environment 204 can combine synthetic trends data received from each of the environments 202a-b while mitigating risk of disclosing personally identifiable information (PII) or other sensitive information related to individuals and entities represented in the combined data. In some cases, the federated cleanroom environment 204 is implemented as a TRE/SPE. The TRE/SPE enforces role-based access control, purpose binding, rate limiting, and comprehensive audit logging, and admits only privacy-enhanced artifacts (e.g., synthetic health trends and synthetic consumer trends embeddings and linkable ground-truth trends). Downstream components consume these artifacts via policy-enforced interfaces so that originating environment controls remain in effect.

A model training environment 206 can receive the combined synthetic trends data from the federated cleanroom environment 204 and train one or more machine learning models using the combined synthetic trends data as training data.

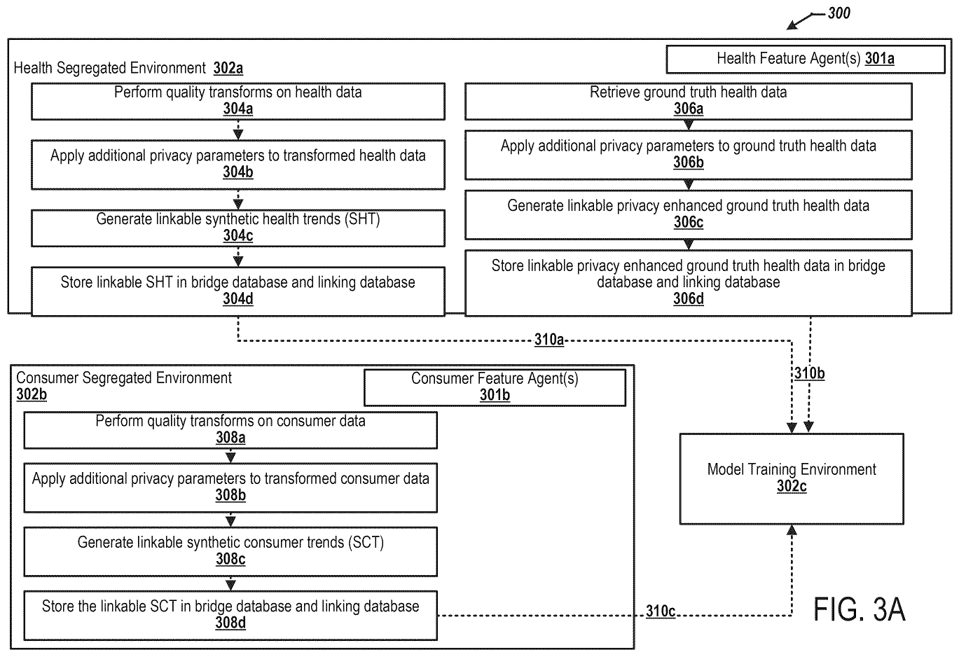

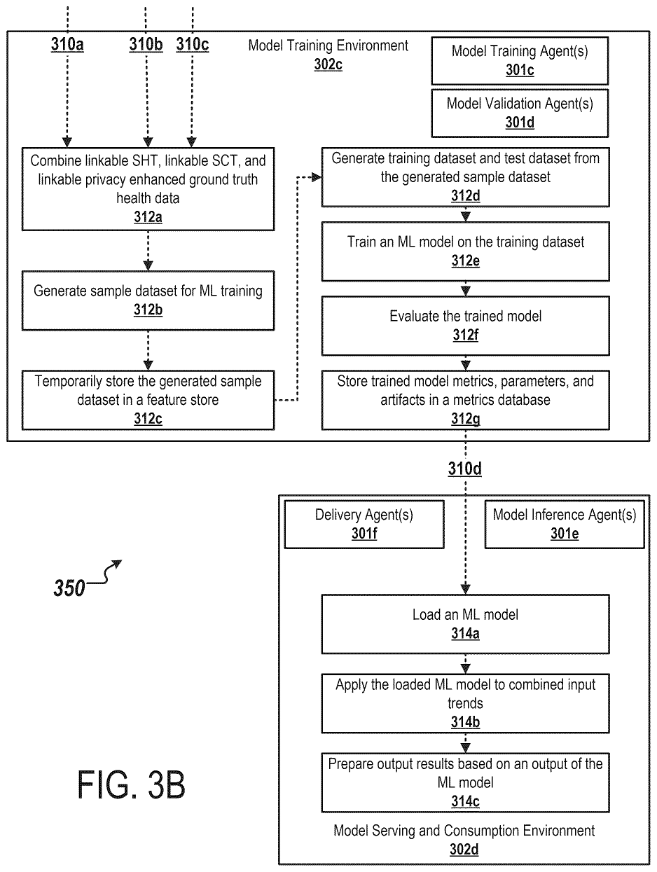

An analytics environment 208 (e.g., a model serving environment) can receive a trained machine learning model from the model training environment 206. The analytics environment 208 can include one or more servers (e.g., a distributed cloud based systems) with one or more processors configured to execute instructions associated with the trained machine learning model. In some implementations, the trained machine learning model is configured to receive inputs from a user or another data source, process the received inputs to generate output data. The output data can be consumed at a consumption layer 210, e.g., via a user interface of a user device. Further detail related to each of the environments described in relation to FIG. 2 is provided below in relation to the description of FIG. 3.

FIG. 3A illustrates an example process 300 for generating a trained machine learning model trained on synthetic trends data. Various steps of the example process 300 are implemented in particular data environments of an example system, as described in relation to the data environments of the description of FIG. 2.

The system includes a health data environment 302a (e.g., a health segregated data bridge). The health data environment 302a includes one or more processors configured to process health data according to a set of data processing operations. The processors of the health data environment 302a are configured to perform (304a) quality transforms on health data to generate transformed health data and to store the transformed health data in a database located in the health data environment 302a.

The processors of the health data environment 302a are configured to apply (304b) additional privacy parameters to the transformed health data. One or more privacy enhancing techniques can be applied, including dimensionality reduction, noise injection, and data compression.

The processors of the health data environment 302a are configured to generate (304c) linkable synthetic health trends (SHT) data and to store (304d) the linkable SHT data in a bridge database and a linking database. In a general sense, the bridge database is a database that acts as an intermediary between two or more systems. In some implementations, the linkable SHT data include embedded representations of the privacy enhanced transformed health data, in which each record in the linkable SHT data can include a token operable to link the corresponding data to data represented in a different dataset (e.g., in a dataset stored outside of the health data environment 302a). In a general sense, a bridge database stores data within security and compliance boundaries of a particular data environment. A linking database is implemented in a security zone independent of any data environment and stores linkable data artifacts needed to perform cross-environment association, such as salted or keyed tokens and minimal trend features. The linking database excludes raw data sources and trend features that would enable reconstruction of source data.

In addition to performing the steps 304a-c, as described above, the processors of the health data environment 302a processors can perform a set of parallel steps (306a-d). The processors of the health data environment 302a are configured to retrieve (306a) health data for a ground truth health dataset. The ground truth health dataset is a subset of the data stored in the health data environment 302a. The ground truth health dataset can include particular actions (e.g., medications) and outcomes (e.g., health-related outcomes associated with the medications) associated with the data stored in the health data environment 302a.

The processors of the health data environment 302a are configured to apply (306b) additional privacy parameters to the ground truth health dataset to generate a privacy enhanced ground truth health dataset, in which the privacy parameters can be the same as those related to the 304b step described above in relation to processing the full health dataset. The processors of the health data environment 302a generate (306c) linkable privacy enhanced ground truth health data and store (306d) the privacy enhanced ground truth health dataset in the bridge database and the linking database.

In some implementations, the processors of the health data environment 302a are configured to make the privacy enhanced ground truth data linkable (e.g., by associating data elements with tokens associated with particular individuals and/or entities represented in the ground truth data) and to store the linkable privacy enhanced ground truth data in the bridge database and the linking database.

In some implementations, each token is unique to an individual or entity associated with a particular data element and can be used to link the particular data element to other data associated with the same individual or entity stored in other data environments.

The processors of the health data environment 302a can transmit the linkable SHT data and the linkable privacy enhanced ground truth health data to a model training data environment 302c. In some cases, processors of the model training data environment 302c are configured to access the linkable privacy enhanced ground truth health data and the linkable SHT data from a database accessible to the environment 302c.

The health data environment 302a includes one or more health feature agents 301a. The health feature agents 301a can interact with other data environments, perform analytics on data accessible to processors of the health data environment 302a, including privacy analytics, monitoring, feedback, among other operations. In some implementations, the health feature agents 301a interact with analysts performing manual tasks and evaluation tasks associated with the processes implemented by the processors of the health data environment 302a. In some cases, the health feature agents 301a are implementations of an AI system, GenAI system, LLM, or other machine learning applications configured to process data and generate automated outputs.

The system includes a consumer data environment 302b (e.g., a consumer segregated data bridge). The consumer data environment 302b includes one or more processors configured to process consumer data according to a set of data processing operations. The processors of the consumer data environment 302b are configured to perform (308a) quality checks and transforms on consumer data to generate transformed consumer data and to store the transformed consumer data in a database located in the consumer data environment 302b.

The processors of the consumer data environment 302b are configured to apply (308b) additional privacy parameters to the transformed consumer data. One or more privacy enhancing techniques can be applied, including dimensionality reduction, noise injection, and data compression, similar to the privacy enhancing techniques described in relation to the health data environment 302a.

The processors of the consumer data environment 302b are configured to generate (308c) linkable synthetic consumer trends (SCT) data and to store (308d) the linkable SCT data in a bridge database and the linking database. The linkable SCT data include embedded representations of the privacy enhanced consumer data, in which each record in the linkable SCT data can include a token operative to link the corresponding data to data represented in a different dataset (e.g., in a dataset outside of the consumer data environment 302b).

In some implementations, each token is unique to an individual or entity associated with a particular data element and can be used to link the particular data element to other data associated with the same individual or entity stored in other data environments.

The consumer data environment 302b includes one or more consumer feature agents 301b. The consumer feature agents 301b can interact with other data environments, perform analytics on data processed by the processors of the consumer data environment 302b, including privacy analytics, monitoring, feedback, among other operations. In some implementations, the consumer feature agents 301b interact with analysts performing manual tasks and evaluation tasks associated with the processes implemented by the processors of the consumer data environment 302b. In some cases, the consumer feature agents 301b are implementations of an AI system, GenAI system, LLM, or other machine learning application configured to process data and generate automated outputs.

The bridge database of each data environment stores data of the respective data environment. Each bridge database resides within a security and compliance boundary of its respective segregated data environment. Each bridge database is accessible to processors in other data environments through policy-enforced interfaces that apply purpose limitation, rate limiting, and audit logging, thereby preserving the respective data environment's controls even when select data are consumed downstream by a processor of a different data environment. In some embodiments, this security and compliance boundary is provided by a TRE/SPE. The additional privacy parameters along with the representation of health and consumer data as synthetic health trends mitigates a risk of associating particular data elements in each of the SHT and SCT with a particular set of PII or sensitive information. Similar to the health data environment 302a, the consumer data environment 302b processor transmits or otherwise makes accessible the linkable SCT data to the model training data environment 302c. In some embodiments, this security and compliance boundary is provided by a TRE/SPE.

In some implementations, instead of transmitting the data to the model training data environment 302c (e.g., from the environments 302a, 302b), the model training data environment 302c accesses databases to retrieve the data (e.g., the linking database and/or the bridge database).

FIG. 3B illustrates a continuation of the example process 300 illustrated as an example process 350 for generating a trained machine learning model trained on synthetic trends data. Various steps of the example process 350 are implemented in particular data environments of an example system, as described in relation to the data environment of FIG. 2.

As described in FIG. 3A, the model training data environment 302c includes one or more processors configured to execute operations associated with training a machine learning model. The processors of the model training data environment 306 are configured to combine (312a) the linkable SHT data, the linkable SCT data, and the linkable privacy enhanced ground truth health data in a combined dataset.

The processors of the model training data environment 302c are configured to generate (312b) a sample dataset for machine learning (ML) training. In some implementations, the sample dataset is a random sampling of the combined dataset.

The processors of the model training data environment 302c are configured to store (312c), temporarily, the sample dataset in a feature store. In some implementations, the feature store is a repository for storing, managing, and serving ML features, which are individually measurable properties or characteristics used as inputs for ML models.

The processors of the model training data environment 306 are configured to generate (312d) a training dataset and a test dataset from the sample dataset. In some implementations, the training dataset and test dataset are randomly selected from the sample dataset. The processors of the model training data environment 302c are configured to train (312e) an ML model on the training dataset, evaluate (312f) the trained ML model, and store (312g) ML model metrics, parameters, and artifacts in a database located in the model training data environment 302c.

Like the environments 302a-b, the environment 302c includes agents configured to monitor, provide feedback, and analyze intermediate data associated with operations performed by associated processors of the environment 302c. The environment 302c includes a model training agent 301c and a model validation agent 301d.

A model serving and consumption data environment 302d receives or accesses data generated from the model training data environment 302c (e.g., the ML model metrics, parameters, and artifacts) associated with the trained ML model. The model server data environment 302d includes one or more processors (e.g., processors associated with the server 106 described in relation to FIG. 1).

The processors of the model serving and consumption data environment 302d are configured to load (314a) an appropriate ML model associated with a particular project or request. In some implementations, the model training environment 302c facilitates training multiple ML models associated with a variety of applications. The processors of the environment 302d can select the appropriate model to load based on a particular task.

The processors of the model serving and consumption data environment 302d are configured to apply (314b) the loaded trained model on a new set of combined trends (e.g., combined SHT and SCT) data. The new set of combined trends data are inputs to the loaded and processed by the trained model. In some instances, the processors of the environment 302d access data from the health data environment 302a and the consumer data environment 302b to process with the loaded trained model. This step is associated with performing an inference operation associated with the trained ML model. An input data set (e.g., synthetic trends data) are processed by the trained ML model to generate an output data set, indicative of a particular insight (e.g., score, classification, etc.).

The processors of the model serving and consumption data environment 302d are configured to prepare (314c) results based on the output data of the trained ML model. In some implementations, the preparation includes data formatting, generation of data visualizations, among other data processing steps. In some implementations, the processors of the model serving and consumption data environment 302d are configured to package the prepared results (e.g., a final deliverable) for export in an associated format (e.g., a project volume). The process performed by the processors of the model serving and consumption data environment 302d are associated with an implementation of the trained machine learning model, as described by operations performed in relation to environments 302a-c.

The environment 302d includes a delivery agent 301f and a model inference agent 301e that are configured to monitor, analyzer, and perform functions associated with operations of the processors associated with the environment 302d.

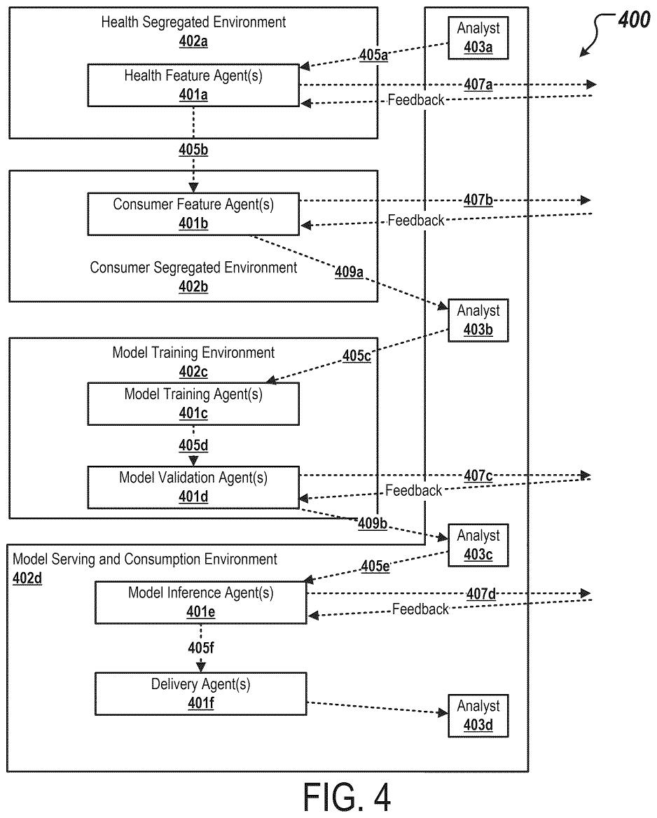

The steps performed by processors of each data environment (e.g., environments 302, 304, 306, and 308) can be performed sequentially, or in parallel when appropriate. Each step can be initiated manually (e.g., by an analyst) or automatically (e.g., in response to a trigger or by an AI agent). The description of FIG. 4, provided below, is a representation of the steps facilitated by AI agents (e.g., by the health feature agent 301a, the consumer feature agent 301b, the model training agent 301c, the model validation agent 301d, the delivery agent 301f, and the model inference agent 301e), executing instructions in each of the data environments and configured to communicate with AI agents in other data environments.

FIG. 4 illustrates an example scenario 400 that includes AI agents 401a-f for performing processes related to generating a trained machine learning model trained on synthetic trends data and executing inferences of the trained machine learning model. The scenario 400 is associated with execution of the processes 300, 350, in which processors associated with data environments (e.g., environments 302a-d) process data to perform various tasks. The scenario 400 illustrates a governance and privacy operations layer in which one or more AI agents 401a-f operate within a respective data environments 402a-d (similar to the data environments 302a-c).

The scenario 400 illustrates communication channels between the AI agents 401a-f and entities 403a-d (e.g., analysts) operating within a model serving and consumption data environment 402d. The AI agents 401a-f can transmit status alert signals, to an analyst operating within the environment 402d. The status alert signals can be associated with a data processing step as described in relation to FIGS. 3A-B. For example, each AI agent 401a-f can generate a status alert related to an audience definition, bias/fairness metrics, dimensionality reduction, flags related to ethical guardrails, and inconsistent data distributions. In some implementations, an agent of the AI agents 401a-f associated with a particular data environment (e.g., a health data environment 402a) communicates with a particular analyst of the environment 402d.

The AI agents 401a-f can transmit human-readable status alerts that encapsulate an outcome of intermediate checks on processes implemented by the AI agents 401a-f. The status alerts can provide sufficient context for oversight of the processes. For example, a health feature agent can generate an alert that states that a particular audience definition surpasses a pre-set uniqueness threshold and recommends either a categorical generalization or variable suppression prior to generation of associated synthetic trends data. As another example, a consumer feature agent can generate an alert that indicates a reconstruction or model inversion risk exceeds a configured privacy or AI-security budget for specific feature combinations and that additional noise injection or discretization has been applied to restore compliance. As another example, a model training agent can generate a status alert to report convergence diagnostics and early-stopping rationales, accompanied by lineage identifiers that bind code commits, feature versions, and sampling seeds. As another example, a model validation agent can generate a status alert indicative of fairness constraints for protected strata that remain within acceptance bands while calibration drift has been detected beyond tolerances. The agent may propose recalibration or feature ablation. As another example, a model inference agent can generate a status alert at runtime indicative of endpoint health, input drift relative to training distributions, and motivated-intruder test outcomes intended to detect prompt or query patterns that could elicit sensitive attributes. Each of these example status alerts are propagated with an associated severity level, provenance tags, and log references to support auditability and rapid remediation.

In some implementations, processors of each data environment can receive a feedback signal, from an analyst operating within the environment 402d in response to the provided alert signal. In some implementations, the feedback signal is processed and leads to a modification of one or more parameters of a respective data processing step, e.g., to modify embedding methods, privacy enhancing techniques, dimensionality reduction approaches, noise addition parameterization, reconstruction risk methods, variable exclusions, among others.

An analyst can provide the feedback as a structured data object through a user interface or a system orchestrator module operating within the environment 402d. The structured data can be processed as control signals by respective agents of each data environment. Example control signals include approval and hold signals that advance or throttle subsequent data processing stages, targeted parameter adjustments that specific revised privacy and AI-security budgets, updated dimensionality reduction hyperparameters, feature exclusion lists, requests to retry a data processing step with alternative sampling strategies, and instructions to roll back the data processing steps to a prior lineage checkpoint. Upon receiving the feedback, an AI agent validates authorization, applies a requested modification to a task configuration, and re-executes an affected data processing step such that outputs, metrics, and lineage records remain unaffected. The AI agents also acknowledge completion of the requested change by emitting a follow-up status alert with updated metrics and cross references to prior data processing steps. In some implementations, certain AI agents accept feedback from other AI agents, enabling a closed loop orchestration system in which, for example, a validation AI agent can instruct a training AI agent to execute hyperparameter tuning within narrowed bounds if prescribed risk and/or performance metric thresholds are not met.

An AI agent can receive a task signal from an analyst operating within the environment 402d that initiates a process to be performed by the AI agent. In addition, the AI agent can receive a task signal from another AI agent. For example, an AI agent operating within the health data environment 402a can receive a task signal from an analyst operating within the environment 402d to perform quality checks on health data and to generate synthetic health trends. As another example, a different AI agent operating within a consumer data environment 402b can receive a task signal from the AI agent operating within the health data environment 402a to perform quality checks on consumer data associated with a particular individual and to generate associated synthetic consumer trends. Other task signals can include instructions for combining segregated data, determining data features, training a machine learning model, validating a machine learning model, and calculating an inference using a trained machine learning model. In some cases, the task signals are initiated by an end user using a user interface, an application programming interface, or based on a schedule of task execution.

The scenario 400 includes an analyst 403a operating within the environment 402d that transmits a task signal 405a to a health feature AI agent 401a operating within the health data environment 402a. The health feature AI agent 401a is configured to perform feature engineering tasks associated with health data, perform quality checks on the health data, convert the health data to SHT data, and to create ground truth data, as according to 304a-d and 306a-d described in relation to FIG. 3A.

The AI agent 401a transmits status alert signal 407a to an entity operating within the environment 402d (e.g., to the analyst 403a or a database within the environment 402d). The status alert signal 407a can include an audience definition, bias/fairness metrics, details related to dimensionality reduction, flags on pre-determined ethical guardrails, and inconsistent data distributions.

The AI agent 401a can transmit the status alert signal 407a to an entity based on a policy-based routing scheme. Status alerts pertaining to sensitive attribute leakage, reconstruction risk thresholds, or model inversion risk thresholds may be routed to an AI governance entity and a privacy operations entity for escalation, while status alerts related to model risk tiering, code lineage divergence, and deployment policy violations may be routed to a model risk management entity or development operations entity. In some implementations, the policy-based routing scheme is enforced through role-based access control and data minimization policies so that each recipient receives only data fields necessary for their role and assigned task. In some implementations, the status alert signal 407a is posted to a durable audit log and to a monitoring dashboard that aggregates health signals across data processing pipelines. In some implementations, recipients can subscript to specific classes of status alerts (e.g., security, machine learning, etc.), severity levels, or projects without providing access to the recipients to the underlying data.

Upon completion of the one or more tasks by the health feature AI agent 401a, the health feature AI agent 401a transmits a task signal 405b to a consumer feature AI agent 401b operating within the consumer data environment 402b. The consumer feature AI agent 401b is configured to perform feature engineering tasks associated with consumer data, perform quality checks on the consumer data, and convert the consumer data to SCT data, as according to 308a-d described in relation to FIG. 3A.

The consumer feature AI agent 401b transmits a status alert signal 407b to an entity operating within the environment 402d that can include noise addition parameterization, reconstruction risk metrics, and variable exclusions.

Upon completing one or more processes, the consumer feature AI agent 401b can transmit results of various processes 409a to an analyst 403b operating within the environment 402d. The analyst 403b transmits a task signal 405c to a model training AI agent 401c operating within a model training data environment 402c. The model training AI agent 401c implements sampling of data features received from the analyst 403b and trains a machine learning model on the sampled data features. Upon completion, the AI agent 401c transmits a task signal 405d to a model validation AI agent 401d, also operating within the model training data environment 402c. The model validation AI agent 401d performs model validation processes in relation to the trained model generated by the AI agent 401c.

The model validation processes performed by the model validation AI agent 401d include processes related to technical, AI security, and governance considerations. The model validation AI agent 401d evaluates generalization performance using test training data sets and cross validation techniques, verifies model calibration through reliability analyses, and confirms that trained model performance exceeds a baseline model performance under matched sampling schemes. The agent 401d can identify data leakage and spurious correlation by testing feature permutation importance, conducting ablation studies, and repeating model training under perturbed data seeds to establish model stability. The agent 401d can evaluate fairness and bias metrics across protected strata and relevant subpopulations with explicit threshold that, if exceeded, trigger one or more mitigation procedures or trigger additional approvals. Validation processes can quantify metrics indicative of membership inference resistance, reconstruction risk on synthetic trends embeddings data, and consumption of an allotted privacy budget. Validation processes can confirm features including reproducibility by verifying code lineage, feature store versions and artifact hashes to ensure that trained machine learning models can be reconstructed deterministically. Validation processes can also include adversarial robustness evaluations and red team evaluations to detect code injection and invasion patterns relevant to downstream data usage. In some implementations, the model validation AI agent 401d stores all results, decisions, and exceptions in a structed artifacts data store and emits a validation report for review and further access gating.

The model validation AI agent 401d transmits a status alert signal 407c to an entity operating within the environment 402d that can include accuracy/loss curves, code lineage, data lineage (e.g., synthetic trends), model risk tiering, privacy budget thresholds, among other status alerts.

Upon completing the model validation processes, the model validation AI agent 401d transmits results of the model validation processes 409b to an analyst 403c operating within the environment 402d. The analyst 403c transmits a task signal 405e to a model inference AI agent 401e operating within the environment 402d. The model inference AI agent 401e executes model inference tasks including loading the trained machine learning model and applying the trained model to health and consumer trends data. The model inference AI agent 401e can select appropriate models to execute based on user queries. In some implementations, the model inference AI agent 401e is configured to receive requests and data from users and/or other automated systems to generate predictive outputs with trained machine learning models generated by the model training AI agent 401c and validated by the model validation AI agent 401d.

The model inference AI agent 401e transmits a status alert signal 407d to an entity operating within the environment 402d that can include audience tiering, drift detection, model endpoint monitoring, motivated intruder testing outcomes, and output checking data.

Upon completing the model inference tasks, the AI agent 401e transmits a task signal 405f to a delivery AI agent 401f operating within the environment 402d. The delivery AI agent 401f performs operations associated with packaging a final modeling outcome and analysis results for delivery to an analyst 403d operating within the environment 402d.

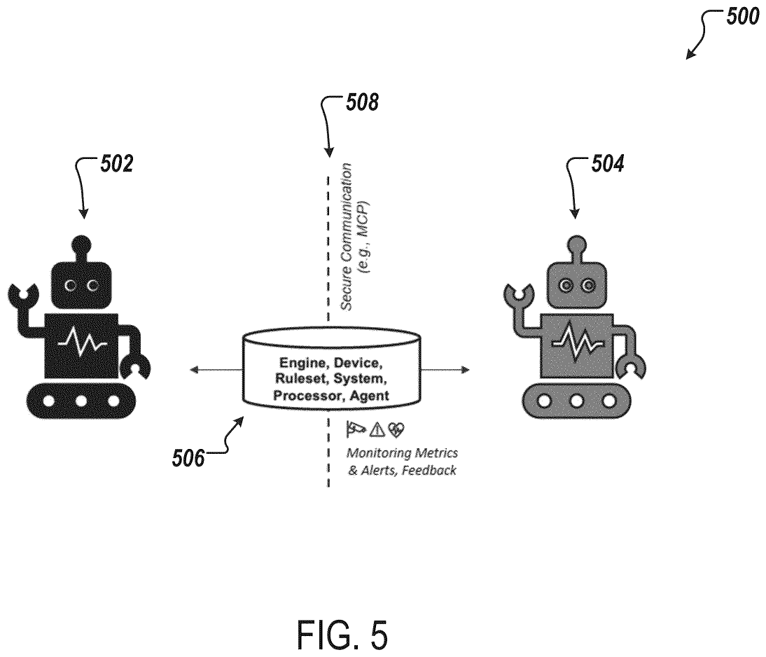

FIG. 5 illustrates an example environment 500 in which a first AI agent 502 and a second AI agent 504 interact under an appropriate secure communication protocol. The first AI agent 502 and the second AI agent 504 can both access a database 506. In addition, the AI agents 502, 504 can access shared engines, devices, rulesets, systems, processors, and functionality of other AI agents. To ensure that AI agents can interact by accessing a common set of resources while mitigating security risks related to prompt injection, malicious code injection, credential leakage, and PII and sensitive information disclosure, a secure communication and data access protocol can be implemented.

The environment 500 represents example circumstances in which the AI agents described in relation to FIG. 4 interact with each other and with external resources.

An example protocol 508 is the Model Context Protocol (MCP). MCP enables structured, secure interactions between AI agents, external tools, APIs, and data sources. MCP provides a standardized framework for AI agents to discover and connect to external sources, execute tool calls (e.g., API requests, database queries, etc.), and to use contextual data to enhance responses generated by LLMs associated with the AI agents. MCP facilitates integrating AI agents into larger computational systems. In some implementations, AI agents register with MCP servers and receive access tokens to use for authorized requests. Other protocols are possible for ensuring proper and secure integration of AI agents into computational systems.

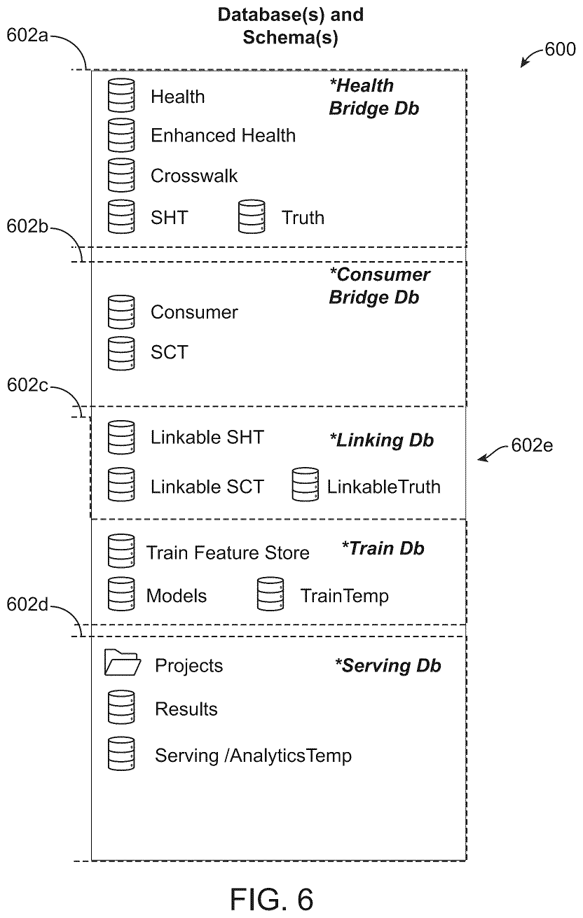

FIG. 6 illustrates example databases 600 implemented in various data environments. The various data environments include a health data environment 602a, a consumer data environment 602b, a model training environment 602c, a model serving and consumption environment 602d, and an external environment 602e.

The health data environment 602a includes a health bridge database with one or more of a health data database, an enhanced health data database (e.g., a database that includes transformed health data), a crosswalk data database, an SHT data database, and a ground truth data database.

The consumer data environment 602b includes a consumer bridge database with one or more of a consumer data database and a synthetic consumer trends data database.

The model training environment 602c includes a training database with one or more of a training features store database, a models database, and temporary training data database.

The model serving and consumption environment 602d includes a serving database with one or more of a projects folder, a results data database, and serving/analytics temporary data database.

The external environment 602e includes a linking database with one or more of a linkable SHT data database, a linkable SCT data database, and linkable ground truth data database. The databases within the external environment 602e include data accessible to any of the processers operating within any of the data environments. Data stored in databases within the external environment 602e are properly anonymized (e.g., SHT, SCT, etc.) such that the data are stored securely.

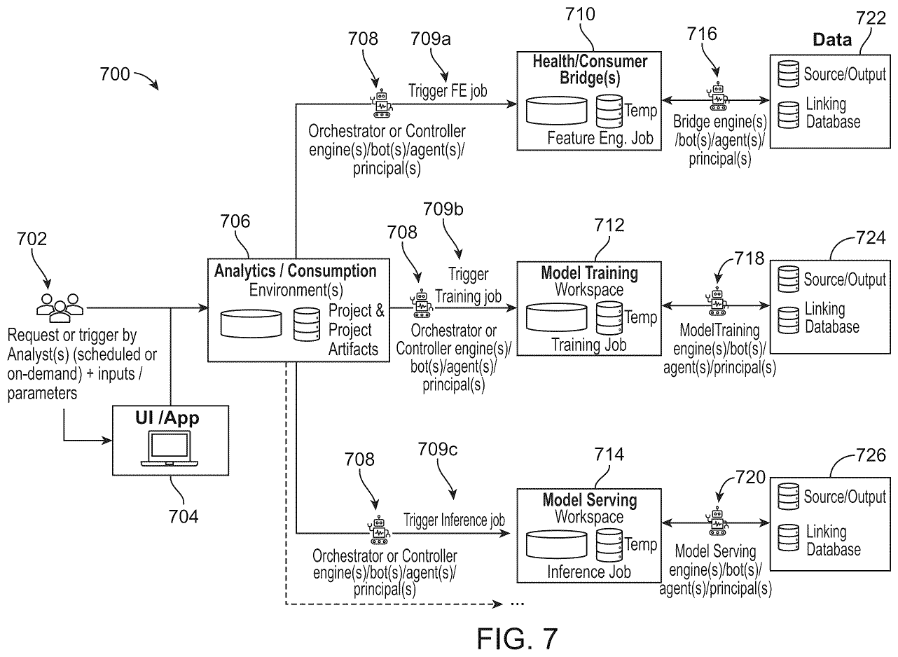

FIG. 7 illustrates an example system 700 for executing various processes with AI agents operating within associated data environments. The system 700 includes a user 702 that interacts with an analytics and consumption environment 706 (e.g., the model serving and consumption data environment 402 as described in relation to FIG. 4) via a user device 704 that includes a user interface or an application interface.

The analytics and consumption environment 706 includes one or more processors configured to execute operations associated with an orchestrator AI agent 708 that is configurable to initiate tasks executed in various data environments. The orchestrator AI agent 708 operating within the environment 706 can interact with external resources and AI agents operating within other data environments. The orchestrator AI agent 708 operating within the environment 706 performs tasks associated with task orchestration and/or task controlling.

As a first example, the orchestrator AI agent 708 can initiate a feature engineering task 708a to be executed by a processor of a health data environment 710. The health data environment 710 can include one or more engines, devices, rule sets, systems, processors, and AI agents. In addition, the health data environment 710 can include one or more temporary databases to store results of various calculations and health data.

The processors of the health data environment 710 implement instructions associated with a health feature agent 716 to access databases 722 that can store source health data, output data from feature engineering processes, and a linking database to link processed health data, synthetic health trends, and data features with data generated by processors of other data environments.

As a second example, the orchestrator AI agent 708 can initiate a training task 709b to be executed by a processor of a model training data environment 712. The model training data environment 712 can include one or more engines, devices, rule sets, systems, processors, and AI agents. In addition, the model training data environment 712 can include one or more temporary databases to store results of model training outcomes (e.g., model weights, training data, etc.).

The processors of the model training data environment 712 implement instructions associated with a model training AI agent 718 to access databases 724 that can store source health and consumer data, output data from feature engineering processes, outputs from model training processes, and a linking database to link associated data between different data environments.

As a third example, the orchestrator AI agent 708 can initiate a model inference task 709c to be executed by a processor of a model serving data environment 714. The model serving data environment 714 can include one or more engines, devices, rule sets, systems, processors, and AI agents. In addition, the model serving data environment 714 can include one or more temporary databases to store results of model inference outcomes (e.g., classifications).

The processors of the model serving data environment 714 implement instructions associated with a model serving AI agent 720 to access databases 726 that can store source health and consumer data, output data from feature engineering processes, outputs from model training processes (e.g., weights), and a linking database to link associated data between different data environments.

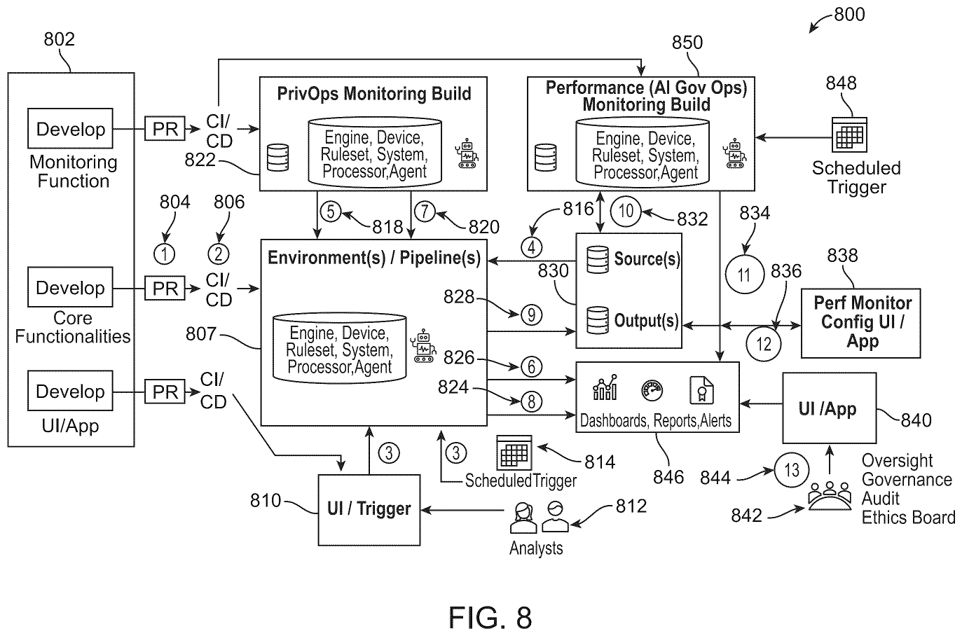

FIG. 8 is an example system 800 for implementing AI governance and privacy operations related to training a machine learning model with data from segregated data environments. The example system 800 includes example components for implementing development monitoring and performing monitoring related to training and implementing machine learning models.

The example system 800 represents an in-line monitoring system in for implementing various monitoring protocols (e.g., privacy monitoring, process oversight, and status/feedback gates, among others). Monitoring protocols ensure performance, privacy, and governance oversight metrics are met. For example, the example system 800 includes a dashboard 846 for monitoring and reporting via a user interface 840 to provide insights 844 related to monitoring metrics, alerts, reporting, auditing functions, and to receive feedback from personnel 842 (e.g., an oversight governance audit ethics board).

In some implementations, the system 800 includes processors operating in data environments to generate metrics and alerts during various stages of development activity operations 802 including during model development (e.g., a CI/CD pipeline 806), model deployment (e.g., source data, input data artifacts, among others), model execution (output data, artifacts, among others), and after multiple instances of model execution to evaluate model drift and other performance metrics. In some implementations, the system 800 monitors various data environments including bridge databases, segregated data environments, AI agents, data linking engines, machine learning modeling agents, and analytics/consumption environments and engines.

Various data processing steps, as described in relation to the description of FIGS. 3A-4, include an interaction between an automated system (e.g., an AI agent) and an end user (e.g., an analyst). In some cases, the automated system receives a task from the end user (e.g., combine segregated data, determine data features, train a machine learning model, etc.). Various tasks (e.g., machine learning model development) can be associated with particular components of the system 800 (the in-line monitoring system).

As an example, with respect to monitoring a model development stage (e.g., development activity operations 802) of model production, the system 800 can monitor pull requests 804 associated with the CI/CD pipeline 806. The pull requests represent how a code base or data might change in relation to developing particular machine learning models. In some cases, the pull requests relate to core functionalities (e.g., data processing), user interfaces, and external applications.

In some cases, a particular monitoring function (e.g., monitoring pull requests) is initiated by an external entity (e.g., analysts 812) via a user interface 810 or a trigger interface. In some other cases, the particular monitoring function is initiated according to a pre-defined trigger schedule 814. The particular monitoring function can be implemented by a processor of a data environment 807 (e.g., the health data environment 302a as described in relation to FIG. 3A). The data environment 807 can include one or more databases, engines, devices, rule sets, systems, processors, agents, and AI agents for executing data processing functionality of the data environment 807 (e.g., determining data features) and monitoring functionality associated with the data environment 807.

The data environment 807 is configured to deliver monitoring metrics related to output data 824 and related to source data 826 to be visually displayed on the dashboard 846. The data environment 807 is also configured to store model artifacts, features, and model outputs 828 in one or more databases 830 that can include output databases (e.g., model outputs) and source databases (e.g., healthcare data databases). The data environment 807 is also operable to retrieve data 816 from the databases 830 (e.g., status checks, and monitoring metrics related to processing source data). The databases 830 can store data from one or more other data environments, e.g., a performance monitoring data environment 850 configured to provide outputs 832 of performance monitoring computations (e.g., model drift) to the databases 830 for storage and access to other data environments. The computations of the outputs 832 can be triggered according to a scheduled trigger 848 or by a trigger initiated by an external entity (e.g., an analyst). Monitoring outputs 834 (e.g., drift monitoring outputs) can also be received by the dashboard 846 for viewing via the user interface 840 by the personnel 842.

The data environment 807 can receive data 818 from a privacy operations monitoring data environment 822 that can include associated monitoring data. In addition to retrieving monitoring data from the environment 822, the data environment 807 is configured to receive monitoring metrics 820 based on output data (e.g., outputs from executing a machine learning model) and model artifacts. A performance monitoring configuration user interface 838 can provide configuration data to be stored in the databases 830 as well as display performance monitoring visualization data 836 on the user interface 838.

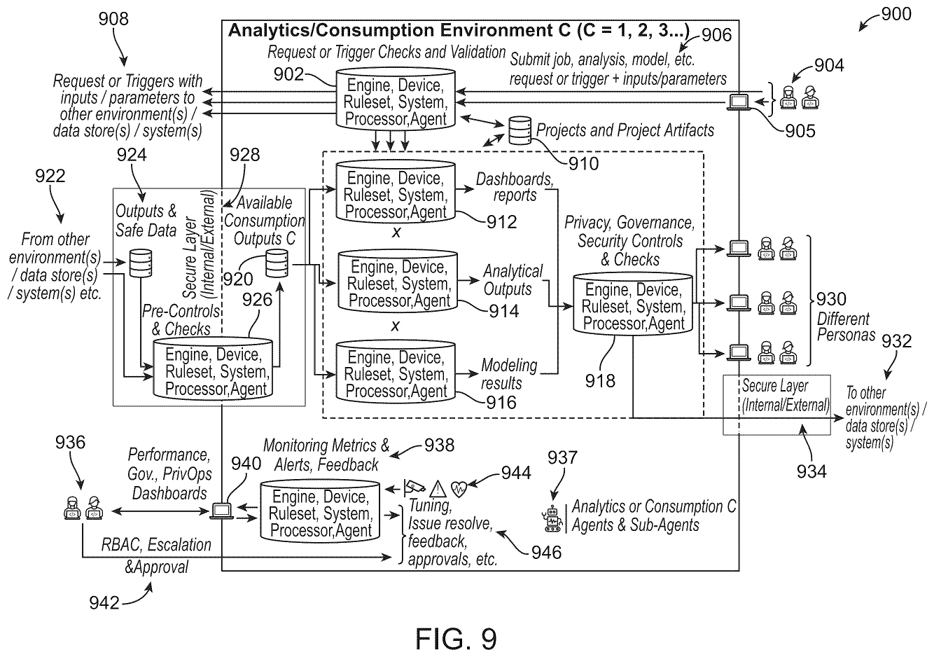

FIG. 9 illustrates an example analytics and consumption environment 900 for monitoring and serving a trained machine learning model. The environment 900 includes one or more processors and components of the environment 900, e.g., AI agents implemented with LLMs, can interact with external resources and AI agents operating within other data environments.

The environment 900 includes multiple processors that can include an implementation of various engines, devices, rule sets, systems, and agents. A request validation processor 902 can receive requests 906 from external entities (e.g., analyst 904). The requests can be indicative of a submitted job to be executed by an AI agent, an analysis, a model to be implemented (e.g., an inference calculation), or a request/trigger with various inputs and parameters, among other possibilities. In some implementations, the request validation processor 902 receives the requests 906 from the analyst 904 via interaction with a user device 905.

The request validator processor 902 processes the requests 906 and determines the type of request. For example, the request validator processor 902 can transmit an output request 908 as the requests 906 or as a secondary request based on the requests 906 with any relevant inputs and parameters to another data environment for further processing (e.g., a health data environment for a feature engineering task related to health data). As another example, the request validator processor 902 can access model projects and model project artifacts from a project database 910, e.g., related to a trained machine learning model, to implement a particular machine learning task. As another example, the request validator processor 902 can transmit the requests 906 to other processors implemented within the environment 900.

The other processors implemented within the environment 900 include a dashboard reports processor 912, an analytical outputs processor 914, a model results processor 916, and a controls processor 918, among other examples. Depending on the nature of the requests 906, the request validator processor 902 can determine a particular processor implemented within the environment 900 to engage.

The processors 912-916 can process data from an available consumption output database 920. The database 920 includes data received from other data environments 922, data stored in a safe data database 924 that meet particular security and privacy thresholds (e.g., synthetic trends data). Before being accessible to the processors 912-916, a pre-controls and checks processor 926 performs one or more security, privacy, and governance checks to data within a secure computing layer 928. In some deployments, the secure computing layer 928 operates within a TRE/SPE so that all analytics-plane processors run inside a governed enclave and only protected insights can exit via egress controls.

In some implementations, prior to making data available to a processor or AI agent operating within an analytics environment (e.g., the analytics and consumption environment 706 described in relation to FIG. 7), the pre-controls and checks processor 926, operating within a secure computing layer to enforce multiple safeguards, verifies data schema and provenance, including digital signatures and lineage identifiers, to ensure only artifacts produced by approved AI agents and pipelines are admitted to the environment. The pre-controls and checks processor 926 can evaluate purpose binding attributes so that data are admitted only for authorized use cases, and records bearing exclusion flags are filtered or transformed. Security controls implemented by the processor 926 can include content sanitization, verification of encryption state and key provenance to ensure at rest and in-transit data security protections. The processor 926 can attach role-based access control manifests to admitted datasets and access control lists are derived from a governance plan accessible to the processor 926. The processor 926 logs all appropriate outcomes (e.g., data admits, data transforms then admits, and data rejects) along with audit entries in a database, in which data entries stored in the database are surfaced on a governance dashboard for oversight functionality.

The dashboard reports processor 912 is configured to generate dashboard data based on data stored in the database 920. For example, the dashboard reports processor 912 can generate graphs, charts, tables, and deliver alerts based on the data stored in the database 920. The analytical outputs processor 914 can generate outputs of calculations based on the data stored in the database 920 (e.g., statistical distributions, averages, sampling, etc.). The modeling results processor 916 can execute instructions associated with a machine learning model (e.g., an inference calculation) to provide an output like a classification, etc. The modeling results processor 916 can access data stored in the database 920 that can include activation data, machine learning parameters, weights, etc.

The controls processor 918 processes outputs from the processors 912-916 to determine various privacy, governance, and security checks before passing the outputs to various personas 930. The personas 930 can include developers, analysts, management, or other stakeholders interested in receiving outputs generated by the processors 912-916 based on the data stored in the database 920. In some cases, the controls processor 918 transmits outputs 932, upon determining the checks, to other data environments via a secure communication layer 934.

In some implementations, various processes implemented by the processors executed within the environment 900 are initiated and monitored by one or more AI agents 937 operating within the environment 900. For example, the execution of instructions associated with the controls processor 918 can be mediated by an AI agent configured to receive output data from the processors 912-916 and to determine the personas 930 and data environments that are relevant to receive the output data.

A monitoring processor 938 can process monitoring data 944 that can include digital surveillance data, alerts, and health data related to data processing steps (e.g., security, privacy, data quality, repeatability, etc.). The monitoring processor 938 can generate monitoring metrics, alerts, and feedback on a performance, governance, and privacy operations dashboard implemented on a monitoring user device 940 accessed by one or more monitoring professionals 936. Based on viewing information displayed on the monitoring user device 940, the monitoring processionals 936 can transmit information 946 to one or more processors operating within the environment 900 that can include role-based access control (RBAC) data, escalation data, and approval data, each associated with particular operations to be executed by a processor within the environment 900. The information 946 can also include tuning data associated with a particular machine learning model and feedback data.

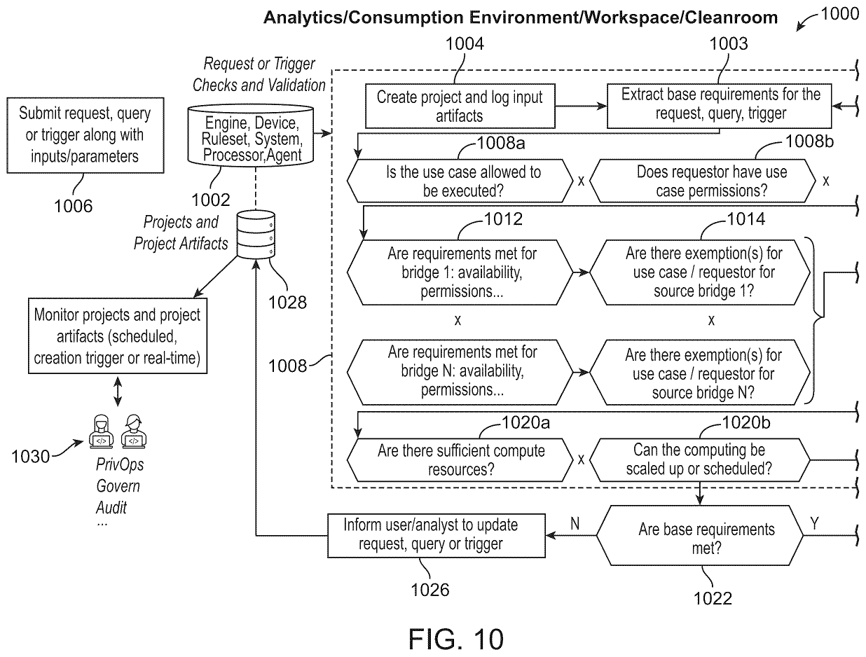

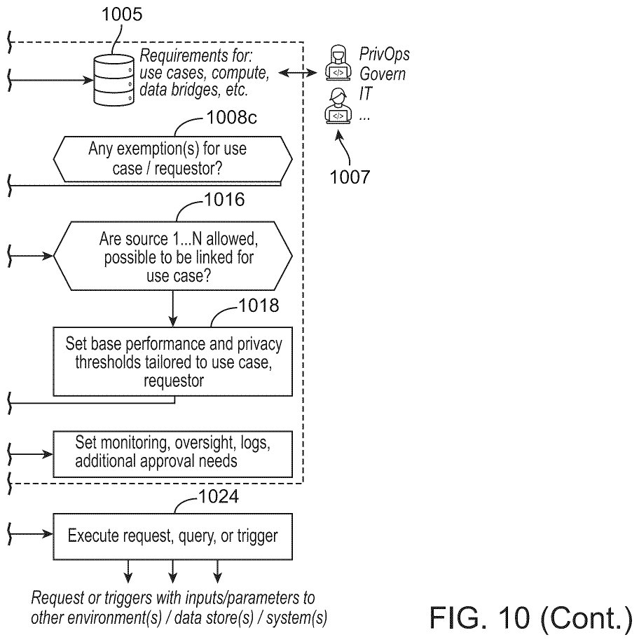

FIG. 10 illustrates an example process 1000 implemented by a processor to determine a task type. The process 1000 can be implemented by a request validation processor 1002 similar to the request validation processor 902 as described in relation to FIG. 9.

The request validation processor 1002 receives a request 1006 that can include query and/or trigger data along with optional input data and other parameters. The request validation processor 1002 creates (1004) a project and logs input artifacts associated with the request 1006. The project can include one or more data files and meta data that describe a task determined by the request validation processor 1002 based on the request 1006. The processor 1002 extracts (1003) base requirements for the request 1006. For example, the base requirements can include information about a particular objective, machine learning model, or application. The processor 1002 stores the base requirements (e.g., use cases, computing resources, data bridges (e.g., to access SHT), among other requirements) in a requirements database 1005. The requirements database 1005 accessible to one or more authorized users 1007 for oversight, governance, and privacy operations.

The processor 1002 determines (1008a) if the use case is allowed to be executed, determines (1008b) if the requestor has use case permissions, and determines (1008c) if there are any exemption(s) for the user case and/or the requestor. For each data bridge that is required, as reflected in the base requirements, the processor 1002 determines (1010) if requirements are met for access to the respective data bridge (e.g., availability, permission, etc.), determines (1012) if there are exemption(s) for the use case and/or request with respect to the respective data bridge. For any data bridge that is eligible (according to (1010) and (1012)), the processor 1002 determines (1014) if the eligible data bridge can be linked to other eligible data bridges. The processor 1002 sets base performance and privacy threshold that are tailored to the use case and the requestor.

The processor 1002 determines (1020a) if there are sufficient compute units based on the base requirements of the use case and determines (1020b) if the computing resources can be scaled up or scheduled to meet the requirements. The processor 1002 can set (1032) monitoring, oversight, logs, and additional approval needs appropriately. The processor 1002 determines (1022) if the base requirements are met. If the base requirements are met, the processor 1002 executes (1024) instructions associated with the request 1006 and transmits the task to a computing component of a data environment. If the base requirements are not met, the processor 1002 informs (1026) the request that the request should be updated and stores the information in a projects and project artifacts database 1028. Data stored in the projects and project artifacts database 1028 is accessible and reviewable to authorized users 1030 (e.g., privacy operations and governance auditors). In some implementations, the authorized users 1030 monitor projects and project artifacts (e.g., schedule tasks and real-time creation triggers).

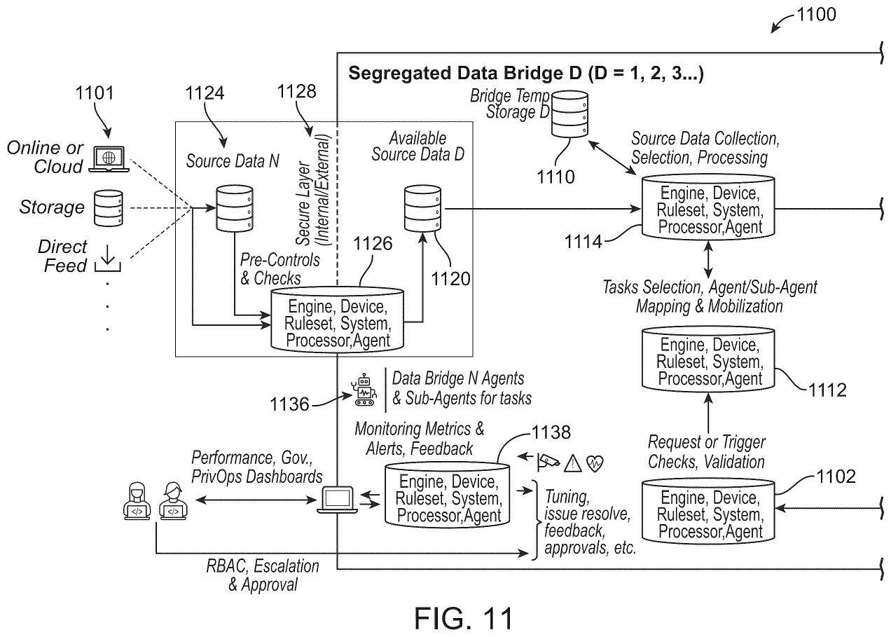

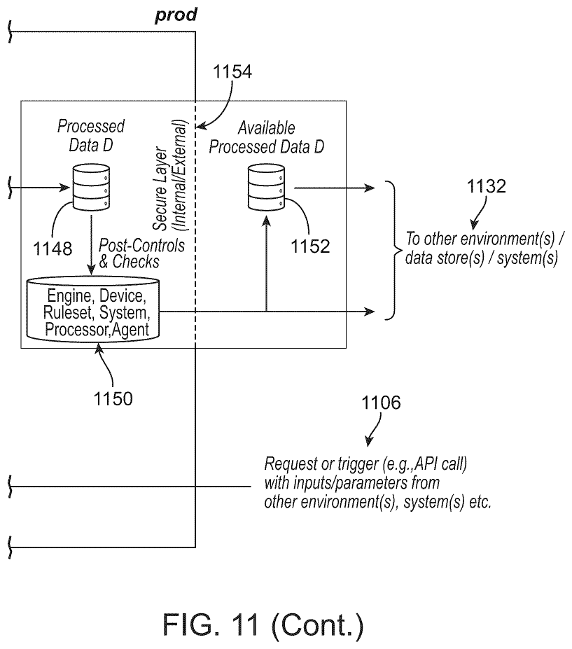

FIG. 11 illustrates an example segregated data environment 1100 (e.g., a health data environment or a consumer data environment) for performing data processing tasks including generating synthetic trends data. The environment 1100 includes one or more processors and components of the environment 1100, e.g., AI agents implemented with LLMs, can interact with external resources and AI agents operating within other data environments.

The environment 1100 includes multiple processors that can include an implementation of various engines, devices, rule sets, systems, and agents. A request validation processor 1102 can receive requests 1106 from external entities (e.g., via an API call from a processor of another data environment). The requests can be indicative of a submitted job to be executed by an AI agent, an analysis, or a request/trigger with various inputs and parameters, among other possibilities.

The request validator processor 1102 can transmit the requests 1106 to a task selection processor 1112. The task selection processor 1112 can implement an AI agent for determining an appropriate task to implement based on the requests 1106 (e.g., feature engineering, synthetic trends data generation, etc.). The task selection processor 1112 can transmit a selected task to a task processor 1114. The task processor 1114 is configured to collect source data, select subsets of the source data relevant to the requests 1106, and processes the subsets of the source data according to the requests 1106.

The processor 1114 can process data from an available source database 1120 (e.g., health data within a health data environment). The database 1120 includes data received from source databases 1124, which can store data received from external data sources 1101 including online or cloud data sources, other databases, and direct data feeds. Before being accessible to the processor 1114, a pre-controls and checks processor 1126 performs one or more security, privacy, and governance checks to data within a secure computing layer 1128. In addition to the available source database 1120, the processor 1114 can access data stored in a bridge database 1110 (e.g., a bridge database that stores SCT data and SHT data). In some embodiments, the secure computing layer 1128 is hosted inside a TRE/SPE that confines computation to the environment and restricts egress to privacy-enhanced outputs.

The processor 1114 generates output data to be stored in a processed data database 1148. For example, the output data can be synthetic health trends associated with the environment 1100 (e.g., SHT or SCT). A post-controls processor 1150 can process data stored in the processed data database 1148 and transmit checked output data through a secure communication layer 1154 to an available processed database 1152 to be consumed by processors of other data environments 1132.

In some implementations, various processes implemented by the processors executed within the environment 1100 are initiated and monitored by one or more AI agents 1136 operating within the environment 1100. For example, the execution of instructions associated with the processor 1114 can be mediated by an AI agent configured to receive output data from the processor 1114 and to determine values of various privacy metrics.

A monitoring processor 1138 can process monitoring data similarly to the monitoring processor 938 as described in relation to FIG. 9.

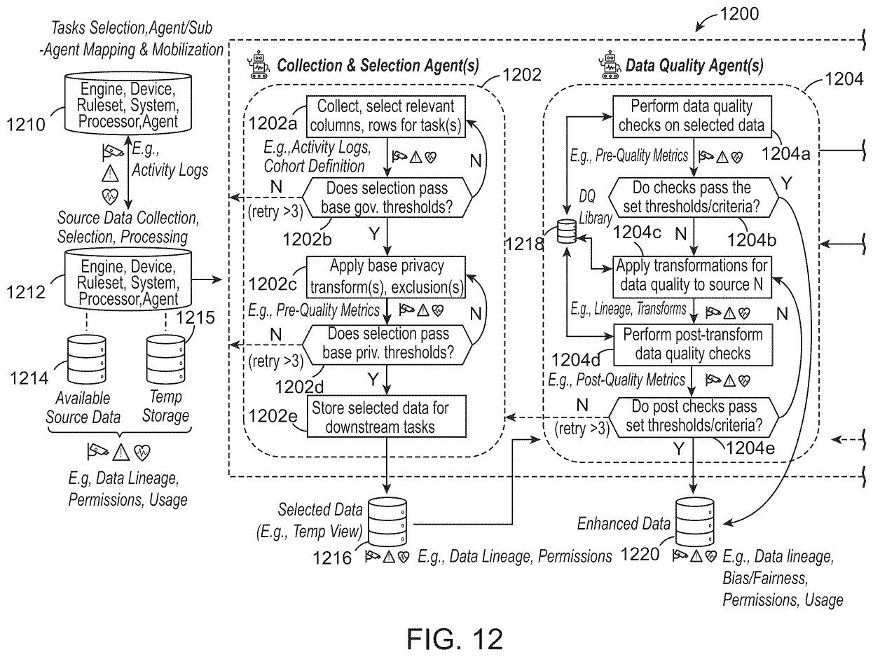

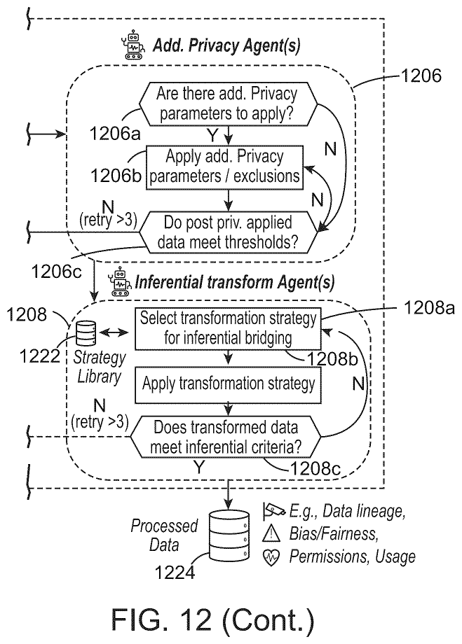

FIG. 12 illustrates a segregated data environment 1200 with AI agents that perform a respective process. For example, the segregated data environment 1200 can be a health data environment or a consumer data environment. The AI agents operating within he segregated data environment 1200 include a collection and selection agent 1202, a data quality agent 1204, an additional privacy agent 1206, and an inferential transform agent 1206. Each AI agent performs one or more data processing tasks related to generating synthetic trends data from source data (e.g., SHT data from source health data).

The collection and selection agent 1202 collects (1202a) and selects relevant columns and rows of source data to perform a particular task. A task selection processor 1210 which operates, in some implementations, outside of the segregated data environment 1200, performs a task selection process. In some implementations, the task selection process includes processing an input request to determine the particular task (e.g., generate synthetic health trends data). A source data collection processor 1212 can receive data from the task selection processor 1210 (e.g., activity logs) to access source data from an available source database 1214 and temporary databases 1215. The source data collection processor 1212 provides the source data to the AI agents of the segregated data environment 1200. For example, the collection and selection agent 1202 collects (1202a) and selects relevant columns and rows of the source data provided by the source data collection processor 1212.

The collection and selection agent 1202 determines (1202b) if the selected columns and rows pass base governance thresholds. If the selected columns and roles do not pass the base governance thresholds, the agent 1202 can return the collection and selection step for a predetermined number of retries (e.g., three). If the predetermined number of retries is exceeded, the agent 1202 can exit the process. If the selected columns and roles pass the base governance thresholds, the agent 1202 applies (1202c) the base privacy transform(s) and exclusion(s). The agent 1202 then determines (1202d) if the transformed selection does not pass base privacy thresholds. If they do not pass the base privacy thresholds, the agent 1202 applies the base privacy transforms again with different parameters until a pre-determined number of retries is met, upon which the agent 1202 exits the process. If the transformed selection passes the base privacy thresholds, the agent 1202 stores (1202e) the selected data for downstream tasks (e.g., machine learning, synthetic trends generation, etc.).

The base governance thresholds define minimum conditions that data selections must satisfy before any downstream processing occurs (e.g., by another agent). In some implementations, the base governance thresholds include limits on high cardinality attribute combinations so that cohorts achieve a required level of indistinguishability. They can include minimum aggregation levels for geographic or temporal dimensions. They can also include mandatory exclusion of fields designated as sensitive or out of scope for a declared purpose. Additional thresholds can be implemented to ensure that contractual restrictions and jurisdictional limitations are respected and that the proposed linkage across environments is permitted for a particular use case. If the threshold are met (e.g., the base governance thresholds), the agent 1202 can proceed to performing quality checks and to the application of additional governance parameters. If the thresholds are not met, the agent 1202 can either retry a process with stricter transforms or terminates the process along with transmitting a status alert for review.

The agent 1202 stores the selected data into a selected data database 1216 that is accessible to the data quality agent 1204. Alternatively or in addition, the collection and selection agent 1202 can pass the selected data to the data quality agent 1204. The data quality agent 1204 performs (1204a) data quality checks on the selected data and stores outputs of the data quality checks in a data quality database 1218 located with the segregated data environment 1200.

The data quality agent 1204 determines (1204b) if the checks pass a set of thresholds and/or criteria (e.g., data quality thresholds). If they do, the data quality agent 1204 stores the selected data in an enhanced data database 1220. If they do not, the data quality agent 1204 applies (1204c) transformations to improve the data quality of the selected data. In some implementations, the data quality agent 1204 stores the transformed data to the data quality database 1218. The data quality agent 1204 performs (1204d) post-transform quality checks and stores outputs of the checks in the data quality database 1218. The data quality agent determines (1204e) if the post-transform data pass the set of thresholds and/or criteria. If they do, the data quality agent 1204 stores the post-transform data into the enhanced data database 1220. If they do not, the data quality agent 1204 passes the selected data back to the collection and selection agent 1202 to revise one or more of the selected data in order to improve the data quality.

The additional privacy agent 1206 receives the selected data that passed the data quality checks performed by the data quality agent 1204. The additional privacy agent 1206 determines (1206a) if there are additional privacy parameters to apply to the post-transform data. If they do, the additional privacy agent 1206 applies (1206b) additional privacy parameters and exclusions to the post-transform data. If they do not, or after the additional privacy agent 1206 applies (1206b), the additional privacy agent 1206 determines (1206c) if the post privacy applied data meet a set of thresholds and/or criteria. If they do not, the additional privacy agent 1206 passes the selected data back to the data quality agent 1204 and/or the collection and selection agent 1202 to revise the selection and/or data quality steps.

The inferential transform agent 1208 receives the post privacy applied data from the additional privacy agent 1206 and selects (1208a) a transformation strategy for inferential bridging. In some implementations, the agent 1208 selects a transformation strategy from a strategy library database 1222. The agent 1208 applies (1208b) the transformation strategy on the post privacy applied data and determines (1208c) if the transformed data meets inferential criteria. If it does, the agent 1208 stores the transformed data in a processed data database 1224. If it does not, the agent 1208 performs one or more retries until it exits this process upon the criteria not being met.

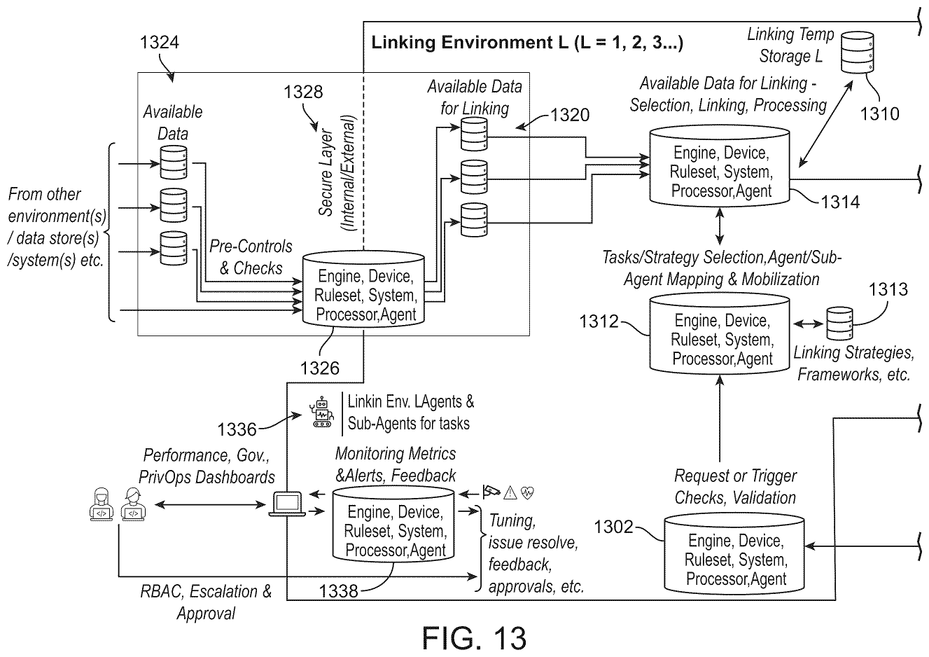

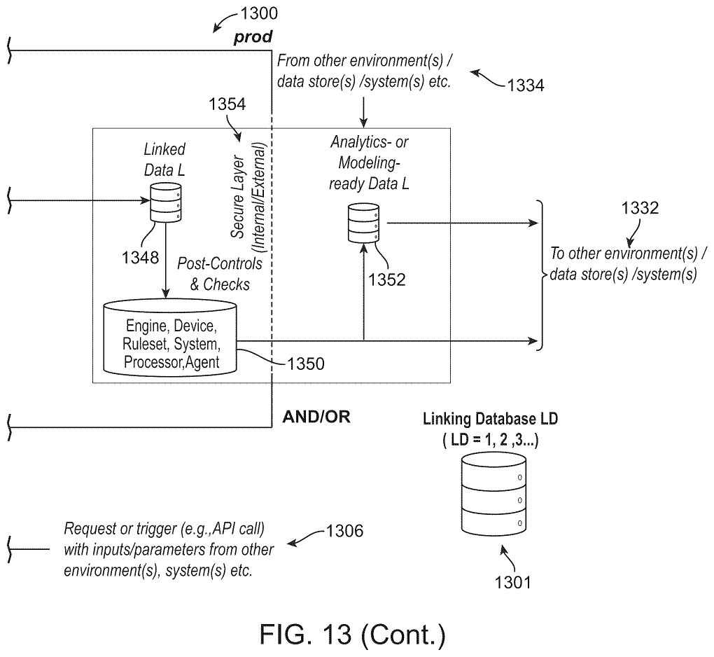

FIG. 13 illustrates an example linking data environment 1300 linking data associated with a particular entity (e.g., an individual) stored in multiple segregated data environments (e.g., a health data environment and a consumer data environment). The environment 1300 includes one or more processors and components of the environment 1300, e.g., AI agents implemented with LLMs, can interact with external resources and AI agents operating within other data environments. As an alternative to the linking data environment 1300, in which links between data elements stored in segregated data environments are determined, the linked data can be stored in a linking database 1301.

The environment 1300 includes multiple processors that can include an implementation of various engines, devices, rule sets, systems, and agents. A request validation processor 1302 can receive requests 1306 from external entities (e.g., via an API call from a processor of another data environment). The requests can be indicative of a submitted job to be executed by an AI agent, an analysis, or a request/trigger with various inputs and parameters, among other possibilities.

The request validator processor 1302 can transmit the requests 1306 to a task and strategy selection processor 1312. The task and strategy selection processor 1312 can implement an AI agent for determining an appropriate task to implement based on the requests 1306 (e.g., linking data stored in a health data environment with data stored in a consumer data environment). The task and strategy selection processor 1312 can access a task and strategy database 1313 that can include linking strategies, frameworks, among other supporting data for performing functionality of the task and strategy selection processor 1312. The task and strategy selection processor 1312 transmits a selected task and/or strategy to a linking processor 1314. The linking processor 1314 is configured to collect data available for linking (e.g., health data, consumer data, etc.) from available databases 1320.

Data stored in the available databases 1320 is sourced from external data 1324 from other data environments, data stores, and systems. Before being accessible to the linking processor 1314, a pre-controls and checks processor 1326 performs one or more security, privacy, and governance checks to data within a secure computing layer 1328. In addition to the available databases 1320, the processor 1314 can access data stored in a linking database 1310.

The linking processor 1314 generates output data to be stored in a linked data database 1348. A post-controls processor 1350 can process data stored in the processed data database 1348 and transmit checked output data through a secure communication layer 1354 to an analytics/modeling ready database 1352 to be consumed by processors of other data environments 1332. In some implementations, linked data from data environments 1334 (e.g., other linking environments) are stored in the analytics/modeling ready database 1352 as well.

In some implementations, various processes implemented by the processors executed within the environment 1300 are initiated and monitored by one or more AI agents 1336 operating within the environment 1300. For example, the execution of instructions associated with the processor 1314 can be mediated by an AI agent configured to receive output data from the processor 1314 and to determine values of various privacy metrics.

A monitoring processor 1338 can process monitoring data similarly to the monitoring processor 938 as described in relation to FIG. 9.

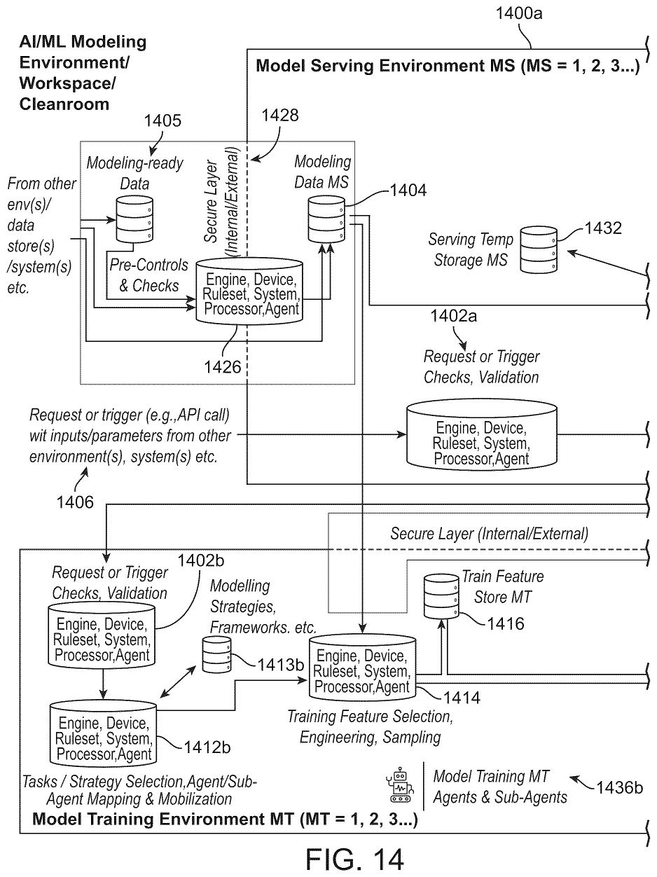

FIG. 14 illustrates an example model serving environment 1400a (e.g., an environment for calculating machine learning model inferences using a trained machine learning model) and an example model training environment 1400b for training the trained machine learning model. The environments 1400a-b each include one or more processors and components of the environments 1400a-b, e.g., AI agents implemented with LLMs, can interact with external resources and AI agents operating within other data environments.

The environment 1400a includes multiple processors that can include an implementation of various engines, devices, rule sets, systems, and agents. A request validation processor 1402a can receive requests 1406 from external entities (e.g., via an API call from a processor of another data environment). The requests can be indicative of a submitted job to be executed by an AI agent, an analysis, or a request/trigger with various inputs and parameters, among other possibilities.

The request validator processor 1402a can transmit the requests 1406 to a task and strategy selection processor 1412a. The task and strategy selection processor 1412a can implement an AI agent for determining an appropriate task to implement based on the requests 1406 (e.g., model training, model inference, etc.). The task and strategy selection processor 1412a can access a model application strategy database 1413a that can include model application strategies, frameworks, among other supporting data for performing functionality of the task and strategy selection processor 1412a. The task and strategy selection processor 1412a can transmit a selected task and/or strategy to a model selection processor 1415 operating within the environment 1400a or a request validator processor 1402b operating within the environment 1400b.

The request validator processor 1402b can transmit the requests 1406 (via the request validator processor 1402a) to a task and strategy selection processor 1412b. The task and strategy selection processor 1412b can implement an AI agent for determining an appropriate task to implement based on the requests 1406 (e.g., model training, model inference, etc.). The task and strategy selection processor 1412b can access a modelling strategy database 1413b that can include modelling strategies, frameworks, training strategies, among other supporting data for performing functionality of the task and strategy selection processor 1412b.

The task and strategy selection processor 1412b transmits selected tasks and/or strategies related to training and designing a machine learning model to a training feature selection processor 1414 that is configured to access modeling data stored in a modeling data database 1404. The modeling data database 1404 stores modeling data from modeling-ready data databases 1405 external to the environments 1400a-b (e.g., the analytics/modeling ready database 1352 as it is described in relation to FIG. 13). Before being accessible to the processor 1402, a pre-controls and checks processor 1426 performs one or more security, privacy, and governance checks to data within a secure computing layer 1428. In certain implementations, the secure computing layer 1428 is deployed as a TRE/SPE, and all tool invocations and model operations occur inside the enclave with dual-approval egress gates. The training feature selection processor 1414 is operable to select training features (variables or combination of variables present in training data) and generate samples of the training data. The training feature selection processor 1414 stores the selected features in a training feature store 1416. The training feature selection processor 141 also transmits or makes accessible via the training feature store 1416 the selected features to a model tuning processor 1418.

The model tuning processor 1418 is configured to perform hyperparameter tuning and model training of a machine learning model as determined by the task and strategy selection processor 1412b and with training data and training features determined by the training feature selection processor 1414. The model tuning processor 1418 accesses a temporary training data database 1420. The model tuning processor 1418 transmits a trained machine learning model to a model validation processor 1422 that also has access to the temporary training data database 1420. The model validation processor 1422 performs validation processes and stores the trained model, model parameters, and metrics in a models database 1424.

Turning to the environment 1400a, the model selection processor 1415 receives a task and/or strategy from the task and strategy selection processor 1412a if the determined task and/or strategy is indicative of a model inference or another implementation of a trained machine learning model. The model selection processor 1415 accesses the models database 1424 to choose an appropriate trained machine learning model with respect to the requests 1406. The model selection processor 1415 also accesses the modeling data database 1404. In some implementations, the model selection processor 1415 applies the trained machine learning model on a combined set of synthetic trends (e.g., SHT and SCT) stored in the modeling data database 1404 and prepares output results for packaging in a deliverable to an end user. In some implementations, the analytics environment outputs audience tiering data, drift detection data, model endpoint monitoring data, motivated intruder testing data, and output checking data.

The model selection processor 1415 performs operations associated with the selected trained machine learning model to generate the output data to be stored in an output data database 1430. The model selection processor 1415 is also operable to initiate a re-training of a trained machine learning model by transmitting an appropriate re-training signal to the task and strategy selection processor 1412a. The model selection processor 1415 has access to a model serving temporary database 1432.

In some implementations, the model serving temporary database 1432 is a temporary storage microservice that provides short-lived or long-lived persistence for intermediate artifacts produced during inference and result preparation. In some implementations, the microservice maintains per-request payloads, feature lookups, transient embeddings, and formatted outputs for a bounded time frame to live sufficiently long to support retries and downstream model packaging by a delivery AI agent (e.g., the delivery agent 301f).

A post-controls processor 1450 can process data stored in the output data database 1430 and transmit checked output data through a secure communication layer 1454 to an available outputs database 1452 to be consumed by processors of other data environments 1456.

In some implementations, various processes implemented by the processors executed within the environments 1400a-b are initiated and monitored by one or more AI agents 1436a-b operating within the environments 1400a-b respectively. For example, the execution of instructions associated with the processors 1412a-b can be mediated by a respective AI agent.

Monitoring processors 1438a-b can process monitoring data associated with respective environments 1400a-b similarly to the monitoring processor 938 as described in relation to FIG. 9.

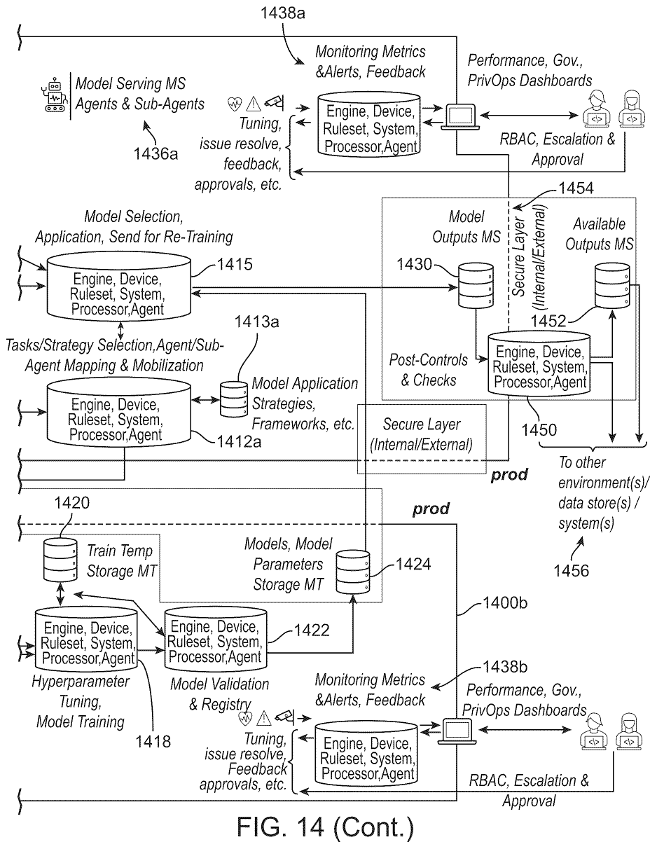

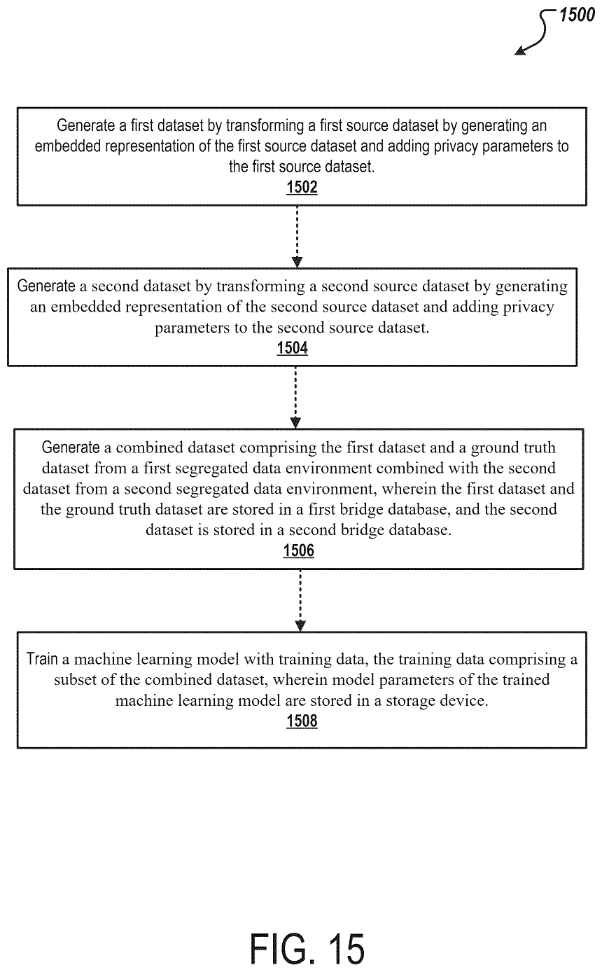

FIG. 15 is a flow diagram of an example process 1500 for generating a trained machine learning model trained on multiple segregated data sources. The example process 1500 can be implemented by a system similar to the systems configured to implement processes 300 and 350 as described in relation to FIGS. 3A-B.

The system generates (1502) a first dataset by transforming a first source dataset by generating an embedded representation of the first source dataset and adding privacy parameters to the first source dataset.

In some implementations, the first source dataset corresponds to health data. In some implementations, the privacy parameters include injected noise.

The system generates (1504) a second dataset by transforming a second source dataset by generating an embedded representation of the second source dataset and adding privacy parameters to the second source dataset.

In some implementations, the second source dataset corresponds to consumer data. In some implementations, the transformation of the first source dataset is performed by a first artificial intelligence (AI) agent operating within the first segregated data environment, and wherein the transformation of the second source dataset is performed by a second AI agent operating within the second segregated data environment. In some implementations, the second AI agent is configured to receive the transformation of the first source dataset from the first AI agent using a model context protocol (MCP) framework of communication between AI agents.

In some implementations, the first dataset and the second dataset each include one or more data elements associated with a shared individual, in which each data element includes a linking key that links a data element of the first dataset with a data element of the second dataset.

In some implementations, the system generates a linking database that includes the first dataset, the second dataset, and corresponding linking keys, in which each linking key is associated with a particular individual.

The system generates (1506) a combined dataset comprising the first dataset and a ground truth dataset from a first segregated data environment combined with the second dataset from a second segregated data environment, wherein the first dataset and the ground truth dataset are stored in a first bridge database, and the second dataset is stored in a second bridge database.

The system trains (1508) a machine learning model with training data, the training data comprising a subset of the combined dataset, wherein model parameters of the trained machine learning model are stored in a storage device.

In some implementations, the system selects a model training strategy from a strategy library database and trains the machine learning model according to the selected model training strategy. The strategy library database includes multiple model training strategies.

In some implementations, training the machine learning model is performed by a model training artificial intelligence (AI) agent operating within a model training environment, wherein the model training environment is different from the first segregated data environment and different from the second segregated data environment.

In some implementations, the system validates the trained machine learning model by a model validation AI agent. The validation includes evaluating the trained machine learning model based on a training dataset. The model validation AI agent is configured to receive model parameters of the trained machine learning model. In some implementations, the validation includes verifying calibration of predicted probabilities, detecting data leakage by retraining the trained machine learning model with perturbed features, assessing fairness across protected strata according to configured bias metrics, and generating a validation report that records metrics, decisions, and exceptions for gating subsequent deployment of the trained machine learning model.

In some implementations, the system transmits, from the model validation AI agent to an entity operating within a model serving data environment, results of the validation process. In some implementations, the system stores the model parameters of the trained machine learning model in a storage device within a model serving data environment. In some implementations, the system receives, at a model inference AI agent operating within the model serving data environment, a task signal from the entity operating within the model serving data environment. The task signal initiates a model inference process performed by the model inference AI agent. In some implementations, the model inference AI agent loads the model parameters of the trained machine learning model from the storage device within the model serving environment to perform the model inference process. In some implementations, the model inference AI agent transmits the results of the inference process to a delivery AI agent operating within the model serving data environment. The delivery AI agent is configured to package results of the inference process for consumption by a second entity operating within the model serving data environment.

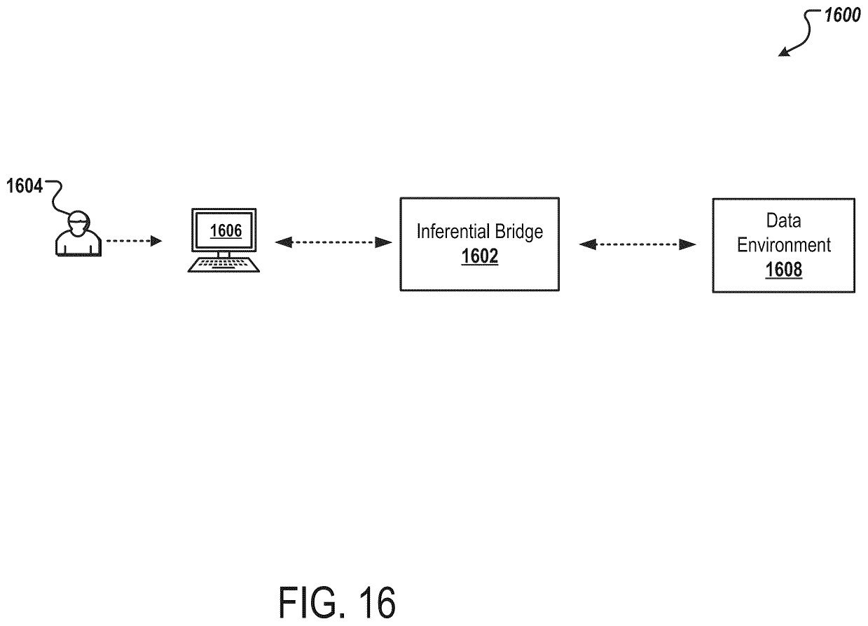

The description provided in relation to FIGS. 1-15 relate to storing, processing, combining, and analyzing data stored in segregated data environments. In some implementations, the systems and methods described in relation to FIGS. 1-15 are performed, at least in part, by AI agents, and include data processing tasks like data validation, generation of embedded representations, training machine learning models, among other data processing tasks. In some implementations, an end user that does not possess permission to access data directly (e.g., due to regulations, privacy concerns, etc.), can access and analyze data stored in segregated data environments through a process referred to as inferential bridging. The following description in relation to FIGS. 16-27 relate to inferential bridging.

Inferential bridging is a method for making inferences from data while preserving confidentiality and privacy of the data. In some cases, the inferential bridging method includes evaluating distributional properties of the data to ensure that insights drawn from the data are protected from a privacy and confidentiality point of view and yield truthful and accurate insights. Inferential bridging can also be viewed as an entry point to a workbench for various applications and services to access insights derived from private and/or confidential data.

The description provided below in relation to inferential bridging concerns an evaluation of an amount of information, which can be interpreted as an amount of knowledge about an object, fact, event, thing, process, idea, notion, etc. In addition, inferential bridging concerns data, which is a narrower concept than information. Data is, e.g., a formalized representation of information prepared for communication, interpretation, and automatic processing by a computing system. As such, data is, for instance, a representation of facts for the purpose of analysis, in which the facts are a representation of an amount of information. Data, in a general sense, can include one or more records, which refers to a set of attributes concerning a single data principal (e.g., a person or an organization). A dataset, in a general sense, can includes a collection of data (e.g., including a collection of records). Inferential bridging allows for analyzing and processing confidential and personal information about data principals and can facilitate a generation of accurate (e.g., truthful) insights and aggregations of data.

FIG. 16 illustrates an example system 1600 that includes an inferential bridge 1602 between an end user 1604 and an environment 1608. The environment 1608 can include multiple segregated data environments. In some implementations, the end user 1604 interacts with the inferential bridge 1602 via a user device 1606 that is communicatively coupled with the inferential bridge 1602.

In some implementations, the inferential bridge 1602 is implemented by one or more processors of one or more servers, and includes at least one networking interface that facilitates a communicative coupling between the one or more servers and the user device 1606.

In some cases, the end user 1604 desires to access data or to determine a statistical output based on data stored in the data environment 1608. However, the end user 1604 may not possess the required credentials or authorization to access the data stored in the data environment 1608. As such, the inferential bridge 1602 provides an access point, e.g., a workbench, for the end user 1604 to extract insights (e.g., inferences, statistical outputs, etc.) and to develop data processing pipelines based on the data stored in the data environment 1608 while preserving privacy and confidentiality of the data. In some implementations, the workbench executes inside a TRE/SPE so that user-submitted code interacts only with protected interfaces exposed by the inferential bridge.

The inferential bridge 1602 evaluates distributional properties of data stored in the data environment 1608 to ensure provided insights maintain privacy and confidentiality and to ensure that transformed data remains an accurate representation of the stored data. Distributional properties are, e.g., mean, variance, skewness, among others. Operations of the inferential bridge 1602 provide accurate statistical inferences, an ability to be integrated into existing analytical systems without extensive modifications to the existing analytical systems, and an ability to ensure confidentiality and privacy of the data by determining data transformations based on data distributions.

In comparison with techniques like differential privacy, inferential bridging generates insights based on distributions of data rather than modified analytical methods. The data stored in the data environment 1608 can include data associated with a data principal, which can be any entity to which the data pertains. For example, a data principal can be a person, organization, device, or a software application. As such, inferential bridging can apply confidentiality (e.g., protecting company secrets) and privacy (e.g., protecting information about people). Various methods of removing sensitive or private information can apply to the inferential bridging process implemented by the inferential bridge 1602 including confidentialization, disclosure control, anonymization, deidentification, and depersonalization, depending on the nature of the data stored in the data environment 1608 and the nature of a request made by the user 1604.

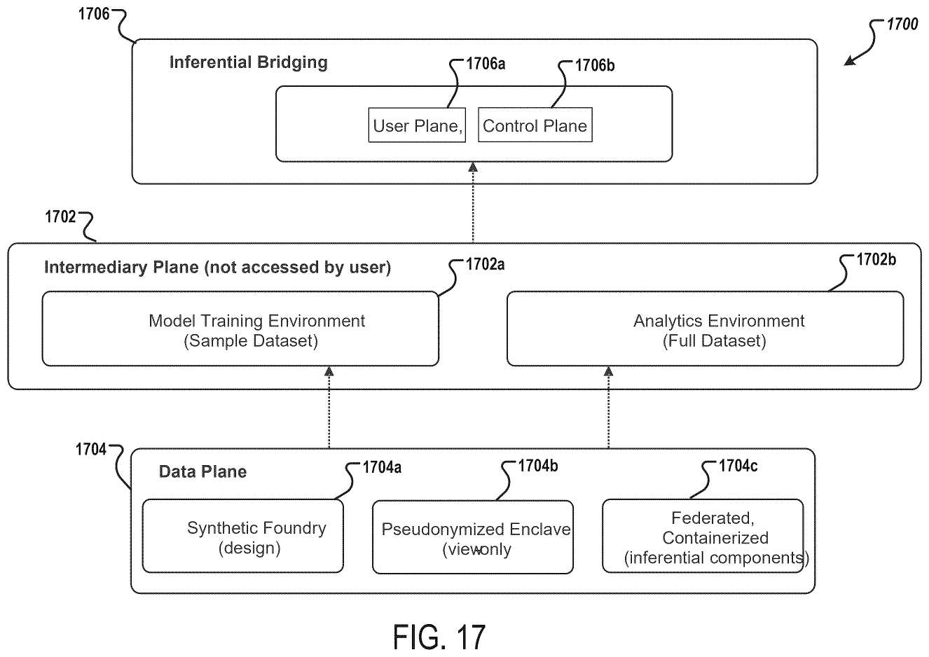

FIG. 17 illustrates an example system 1700 that incorporates features of the model training environment 206 and the analytics environment 208, as described in relation to FIG. 2. In addition, the system 1700 includes features related to inferential bridging as described above, and several data access modalities.

The system 1700 includes an intermediary plane 1702, a data plane 1704, and an inferential bridge 1706. The inferential bridge 1706 includes a user plane 1706a and a control plane 1706b. The intermediary plane 1702, which includes functionality not accessible by an end user, includes a model training environment 1702a and an analytics environment 1702b. The data plane 1704 includes a synthetic foundry access mode 1704a (to deliver data in a synthetic data access mode), a pseudonymized enclave 1705b (to deliver data in a pseudonymized data access mode), and federated and containerized data access mode 1704c (to deliver data in a federated data access mode).

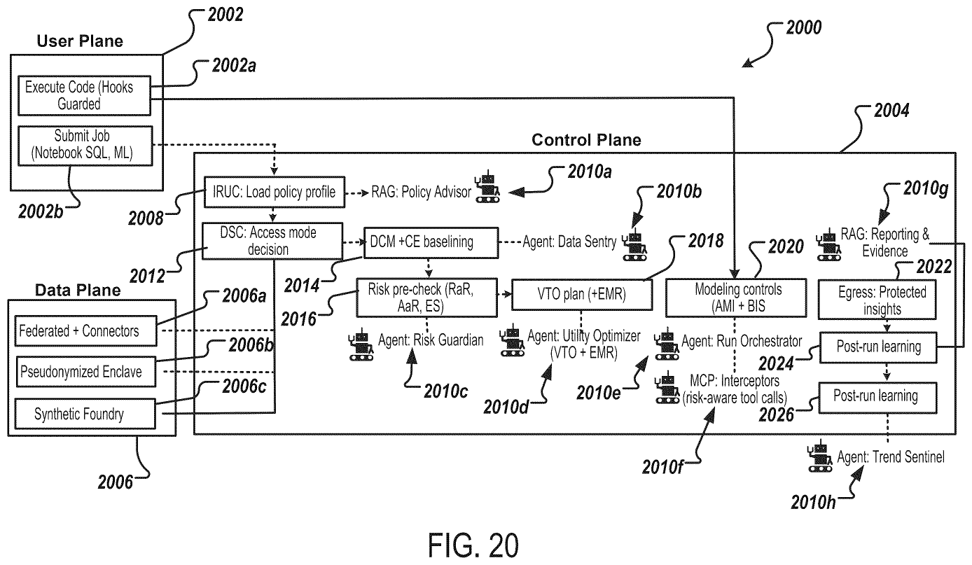

Regarding components of the inferential bridge 1706, the user plane 1706a includes user-facing interfaces for interacting with data stored in the data plane 1704. For example, the user plane 1706a can be an implementation of a Jupyter Notebook, an API endpoint, a database query language interface, among other data access methods. In some implementations, a user interacts with the user plane 1706a via a user interface and implements software code or other series of executable instructions to perform a data analytics task, e.g., training a machine learning model on data stored in a database that resides in and is managed by resources associated with the data plane 1704. In some embodiments, the pseudonymized enclave access mode 1704b (and, in certain deployments, the federated and containerized access mode 1704c) is implemented as a TRE/SPE that confines computation and restricts egress to protected insights. The control plane 1706b includes services and functionality related to ensuring that the data stored in the data plane 1704 is delivered to the user plane 1706a according to relevant governance and privacy protocols and requirements, e.g., operations associated with inferential bridging. Further description related to specific implementations of the control plane 1706b is provided in relation to FIG. 20.

Regarding components of the intermediary plane 1702, the model training environment 1702a can include multiple AI agents that perform tasks related to training a machine learning model. In some implementations, an AI agent trains the machine learning model on a sample dataset. In some implementations, the AI agent trains the machine learning model on a subset of a training data set. Details related to functionality performed within the model training environment 1702a and associated AI agents is provided in relation to FIG. 3B. The analytics environment 1702b can also include multiple AI agents that perform tasks related to generating insights and inferences using one or more trained machine learning models. For example, an AI agent operating within the analytics environment 1702b can processes a subset of data stored in the data plane 1704 with a trained machine learning model. An output of the machine learning model can be delivered to a user in the user plane 1706a to be consumed by a user. Details related to the functionality performed with the analytics environment 1702b and associated AI agents is provided in relation to FIG. 4.

Regarding components of the data plane 1704, each data modality provides data to a user depending on particular tasks to be performed by the user and relevant data access protocols. For example, the synthetic foundry access mode 1704a provides a design-time shadow dataset with matched distributions of a full target dataset for feature engineering. The pseudonymized enclave access mode 1704b provides a view-only workspace (e.g., no data extracts) for hands-on preparation of data (e.g., training data) if fidelity of data provided in the synthetic foundry access mode 1704a is insufficient. The federated and containerized data access mode 1704c provides data for execution-time inferential-only data access (e.g., data outputs generated by a trained machine learning model). In some implementations, a particular user accesses data in the data plane 1704 using each of the data access modes 1704a-c depending on a stage of development. For example, the synthetic foundry access mode 1704a is useful during machine learning model design (e.g., feature engineering), the pseudonymized enclave access mode 1704b is useful for testing an intermediate trained machine learning model, and the federated and containerized access mode 1704c is useful for implementing the full functionality of the trained machine learning model.

Components of the intermediary plane 1702 access and process data stored in the data plane 1704 according to a particular data access mode. The inferential bridge 1706 processes outputs from the intermediary plane 1702 to ensure the outputs provided to a user via the user plane 1706a are confidentialized and secure according to particular protocols and governance requirements, as determined and executed by components of the control plane 1706b.

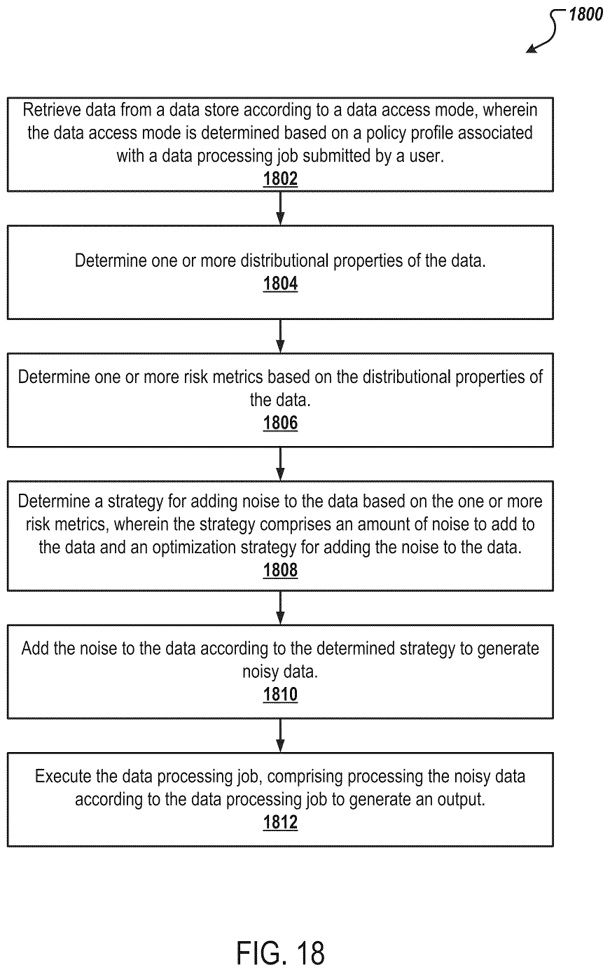

FIG. 18 is a flow diagram of an example process 1800 for implementing inferential bridging. The example process 1800 can be implemented by a system similar to the system described in relation to FIG. 16.

The system retrieves (1802) data from a data store according to a data access mode. The data access mode is determined based on a policy profile associated with a data processing job submitted by a user (e.g., the user 1604 as described in relation to FIG. 16). In some implementations, the system loads the policy profile associated with the data processing job submitted by the user and determines the data access mode based on the data processing job and the policy profile. In some implementations, the policy profile defines a risk budget associated with the data processing job.

In some implementations, the data access mode is a synthetic data access mode that includes delivering synthetic data to the user. In some implementations, the data access mode is a pseudonymized data access mode that includes providing a view-only data access to the user. In some implementations, the data access mode is a federated data access mode that includes delivering protected insights to the user, in which the protected insights are derived from noisy data.

The system determines (1804) one or more distributional properties of the data. In some implementations, the distributional properties of the data include statistical properties of the data, e.g., mean, variance, skewness, etc. In some implementations, the system determines a calibration error of the retrieved data and modifies the retrieved data based on the determined calibration data.

The system determines (1806) one or more risk metrics based on the distributional properties of the data. In some implementations, the one or more risk metrics include records at risk, attributes at risk, and expected shortfall.

The system determines (1808) a strategy for adding noise to the data based on the one or more risk metrics. The strategy includes an amount of noise to add to the data and an optimization strategy for adding the noise to the data. In some implementations, the optimization strategy includes a risk-first strategy. In some implementations, the optimization strategy includes a utility-first strategy. In some implementations, the optimization strategy includes a balanced strategy that includes a risk threshold and a utility threshold. In some implementations, the system logs the determined strategy for adding noise in a provenance log.

In some implementations, the system performs a record-level balancing of the data. The record level balancing includes modifying a number of records from the data associated with a particular classification. In some implementations, the system performs an algorithm-level balancing of the data that includes modifying classification weights of a machine learning model. The classification weights are associated with a particular classification record.

In some implementations, as part of the determined strategy, the system performs a principal component analysis of the data to determine multiple dimensions that characterize the data, in which the multiple dimensions represent a subset of dimensions with the highest variance. The system adds the noise to the data along the multiple dimensions (with the highest variance).

The system adds (1810) the noise to the data according to the determined strategy to generate noisy data. In some implementations, the system determines one or more distributional properties of the noisy data and evaluates one or more updated risk metrics based on the distributional properties of the noisy data, similar to the step described above in relation to the distributional properties of the retrieved data. In some implementations, the system determines that the one or more updated risk metrics exceed a risk budget, in which the risk budget is defined in the policy profile. Responsive to determining that the one or more updated risk metrics exceed the risk budget, the system updates the strategy for adding noise to the data based on the one or more updated risk metrics.

The system executes (1812) the data processing job, which includes processing the noisy data according to the data processing job to generate an output. In some implementations, the system adds noise to the data according to the updated strategy to generate updated noisy data and executes the data processing job, which includes processing the updated noisy data according to the data processing job to generate an updated output.

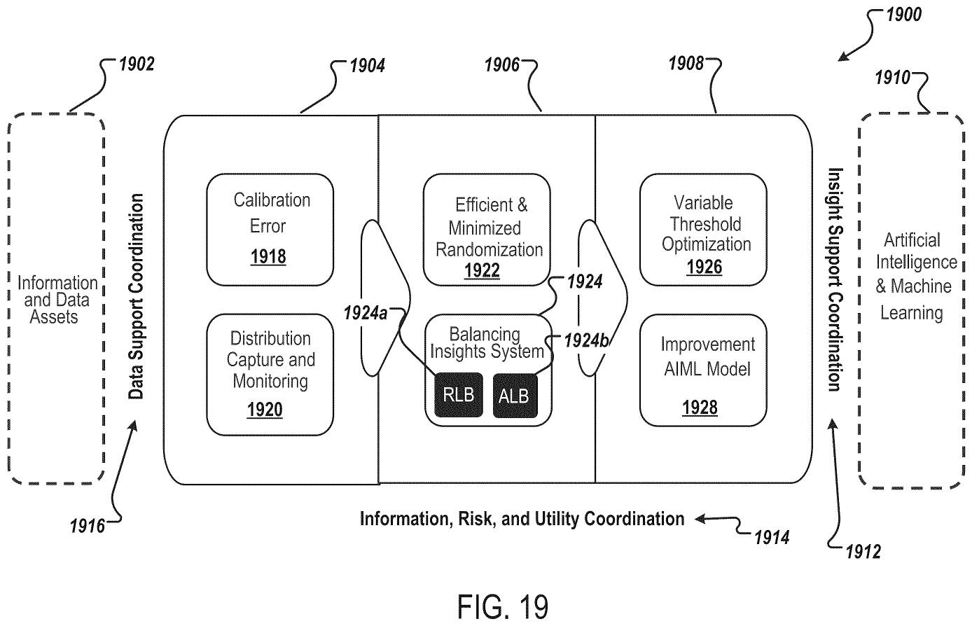

FIG. 19 illustrates an example process 1900 that includes a transformation of information and data assets 1902. The example process 1900 includes an inputs and pre-processing stage 1904, a modeling stage 1906, and an outputs and postprocessing stage 1908. The stages 1904-1908 result in a transformation of the information and data assets 1902 into output data suitable for processing by artificial intelligence and machine learning applications 1910. The transformation ensures appropriate confidentialization and privacy of the assets 1902 according to governance protocols, in which the transformation is implemented by components that execute data processing steps associated with the stages 1904-1908. In some cases, the example process 1900 is executed by a system referred to as an “inferential bridging watchtower” or an “insight sentry.”

The inferential bridging watchtower (e.g., modules that execute the stages 1904-1908) is a control plane (e.g., the control plane 1706b) and operates as an “always-on sentry” that sits between protected information (e.g., the information and data assets 1902) and protected insights (e.g., outputs of the artificial intelligence and machine learning applications 1910), continuously coordinating how information is ingested, transformed, monitored, and released. The inferential bridging watchtower is an example of an operational manifestation of the “inferential bridge,” as described in relation to FIG. 16.

The stages 1904-1908 include receiving the information and data assets 1902 via a data support coordination (DSC 1916) process. The DSC 1916 allocates data access modes (e.g., synthetic data, pseudonymized enclave data, and federated data) according to governance policy and operational need, as described in relation to FIG. 17. The DSC 1916 facilitates a retrieval of data from the information and data assets 1902 according to a data access mode, where the data is to be processed by the stages 1904-1908.

The inputs and pre-processing stage 1904 includes a determination of calibration error (CE 1918). The CE 1918 is evaluated as an inherent uncertainty present in the data. The determination of the CE 1918 includes capturing implicit, inferred, and verification errors to improve both risk estimation and utility estimates. The CE 1918 is treated as part of an empirical distribution of data provided by the DSC 1916, which can be used for informing methods of determining risk metrics and data transformation choices so that protected outputs (e.g., outputs of the artificial intelligence and machine learning applications 1910) are both safe and statistically meaningful. The CE 1918 can be interpreted as the inherent error represented in the information and data assets 1902.

Due to variations in information extraction, collection, synthesis, simulation, as well as unknown parameters, and forms of data missingness (e.g., random, semi-random, not random), data are a form of imputation from knowledge that is captured. As such, data typically has implicit uncertainty due to a variety of error sources that are cumulatively captured in the CE 1918. The inputs and pre-processing stage 1904 can include an estimate of the CE 1918 using parametric and non-parametric statistical modeling.

The inputs and pre-processing stage 1904 includes a distribution capture and monitoring (DCM 1920) process. The DCM 1920 includes determining distributional properties (e.g., potentially from containerized and federated data) from data and monitors the distributional properties for drift. Because inferential bridging includes a transformation of data (e.g., noise is added to the data), preserving unmodified statistics and data processing queries (e.g., user code) is possible while protecting confidentiality and privacy at the bridge boundary. This technique provides continuous utility surveillance (e.g., ensuring outputs are accurate and useful) and enables truthful inference (e.g., valid confidence intervals), distinguishing it from mechanism-defined protections (e.g., differential privacy, which includes a modification of particular functions rather than a modification of the data processed by the functions). Example distributional properties include statistical moments of the data (e.g., mean, variance, and skewness).

The modeling stage 1906 processes data received from the inputs and pre-processing stage 1904 (e.g., after calibration error and empirical distributional properties are determined). The modeling stage 1906 includes an efficient and minimized randomization (EMR 1922) process and an implementation of a balancing insights system (BIS 1924).

The EMR 1922 process can include implementations of dimensionality reduction (e.g., PCA) and clustering. For example, a process like PCA determines dimensions that parametrize the data that have the highest variance. As such, processes associated with the EMR 1922 result in a minimization of an amount of randomization needed by focusing the randomization to data ranges that coincide with high variance and risk, thereby preserving correlations and maximizing usable utility. The EMR 1922 process turns “privacy noise” into a principled, distribution-aware tool aligned to risk thresholds defined in governance and privacy protocols described in relation to the Figures below.

As an example, dimensionality reduction reduces a solution space in which risk metrics are calculated and it scales a transformation of data across attributes to capture correlation between attributes while minimizing the effects of the transformation accordingly. PCA, as an example of dimensionality reduction, reduces the dimensionality of the data through uncorrelated feature extraction. PCA determines an optimal projection of the data based on a direction of greatest variance. By transforming high-dimensional data into an eigenspace allows for an evaluation of a set of highest correlated components of the data. PCA can be applied to summarized information (e.g., summarization of a subset of the information and data assets 1902 after incorporating the CE 1918). Risk metrics are used to evaluate the summarized information, and the eigenvectors provide a weighting to the attributes determined by the PCA for the sake of optimizing transformations (e.g., mapping the determined risk and transformations back into the original information space before PCA was applied). This mapping enables a risk evaluation based on the most important or impactful attributes (e.g., those of highest variance), minimizing the transformations of correlated attributes based on the degree of variation (e.g., a high-variance attribute is transformed more, e.g., with more noise, than those attributes that have less variance, and those attributes with low variance can be ignored based on a practical risk and pre-determined transformation threshold).

The EMR 1922 process can also include an implementation of risk evaluation of the data received from the inputs and pre-processing stage 1904. The risk evaluation can include an evaluation of a records at risk (RaR) metric and an attributes at risk (AaR) metric. In general, the RaR and AaR metrics are based on a value at risk (VaR) framework implemented in financial risk modeling. Both the RaR and AaR metrics are indicative of information at risk (IaR), which summarizes which information about data principals represented in the information and data assets 1902 are at risk (e.g., address, social security number, or other combinations of data fields).

RaR is a risk metric indicative of individual risk profiles (e.g., associated with particular data principals) stored in the information and data assets 1902. The determination of the RaR metric can be performed on transformed data after the inputs and pre-processing stage 1904 and randomization of the EMR 1922 process. In addition, an aggregate RaR risk metric represents a risk profile of an entire dataset with respect to data principals. AaR is a risk metric indicative of a risk profile of a particular attribute (e.g., address, social security number, etc.). An aggregate AaR risk metric represents a risk profile of an entire dataset with respect to data attributes.

Various methods for evaluating the RaR and AaR metrics can be used including parametric modeling and non-parametric modeling (e.g., Monte Carlo). The parametric modeling of RaR includes estimating a risk metric with specified parameters (e.g., correlations, volatility, and risk thresholds) as an input for individual data records. The parametric modeling of AaR includes estimating a risk metric with specific parameters (e.g., correlations, volatility, and risk thresholds) as input for attributes in a dataset or a subset of the dataset. The non-parametric modeling of RaR includes estimating the risk metric by simulating random scenarios and iteratively re-evaluating risk and modifying the parameters. The non-parametric modeling of the AaR includes a similar approach to the non-parametric modeling of the RaR. Parametric modeling approaches are faster and good for estimating linear relationships but are less-accurate for non-linear relationships. Non-parametric modeling approaches are more computationally intensive but are more accurate for non-linear relationships.

The RaR and AaR can each be interpreted as a measure of potential disclosure risk of a set of data records or attributes in a set of data records over a defined period of time for a particular confidence interval. A description of each metric includes a specified degree of risk (e.g., a risk metric), the defined period of time over which the risk is assessed, and the particular confidence interval.

In addition to RaR and AaR, the EMR 1922 process includes an evaluation of an expected shortfall. The expected shortfall is an average risk in a worst case scenario. The expected shortfall, upper bound on RaR, and upper bound on AaR provide a measure of risk for a dataset. In some cases, the expected shortfall is evaluated for a given quantile, defined as a mean loss (e.g., risk of disclosure) below the given quantile. The expected shortfall provides a conservative estimate of an amount of insights that can be drawn from a dataset relative to a risk that is taken by displaying the dataset. By adjusting the quantile that defines the expected shortfall, a different amount of insight and corresponding risk can be achieved.

As an example process for evaluating the risk of a dataset based on RaR and AaR metrics, consider a set of information represented by a combination of multiple datasets. The process includes determining a calibration error and distributional characteristics from the combination of multiple datasets (e.g., mean, variance, etc.). The EMR 1922 process includes determining a minimal amount of noise to be added to the combination of multiple datasets such that risk thresholds are met. The EMR 1922 process includes evaluating risk metrics of a dataset (e.g., a noisy dataset) by evaluating the RaR and AaR. The RaR is evaluated for each individual record associated with each data principal. The AaR is evaluated for each attribute (or a collection) of the dataset across all data principals (or a subset of the data principals, e.g., a cohort). If the RaR and/or AaR risk metrics exceed a pre-determined threshold (e.g., too much risk), the EMR 1922 implements a modified transformation (e.g., implement different randomization with adjusted parameters).

As data and models change, the EMR 1922 process includes a re-evaluation of the risk metrics and compares them to the pre-determined thresholds and baseline values. If the EMR 1922 determines that the RaR and/or AaR do not exceed the pre-determined threshold (e.g., after a number of iterations), the EMR 1922 passes the transformed data (e.g., data with added noise) downstream for processing to derive protected insights. The transformed data allow users (e.g., engineers, data scientists, etc.) to learn about distributions of the data principals and attributes. In some implementations, the EMR 1922 passes noise elements (e.g., a matrix or transformation instructions) downstream for a process to add noise or otherwise transform the data.

In a scenario in which a dataset includes an uneven representation of characteristics (e.g., classes, labels, etc.), the BIS 1924 can be configured to implement a record-level balancing (RLB 1924a) process and an algorithm-level balancing (ALB 1924b) process. The BIS 1924 adjusts proportions of data principals in different groups and cohorts to ensure that insights are not biased toward any particular group or cohort (e.g., demographic, race, etc.). In general, the BIS 1924 adds and subtracts data principals to smooth differences between groups or cohorts.

The RLB 1924a and the ALB 1924b processes rebalance cohorts represented in the data (e.g., the information and data assets 1902) at the record level (e.g., oversample, under sample, or generate synthetic data) or at the algorithm level (class weighting, generation of ensembles, implementation of cost-sensitive learning). The BIS 1924 is implemented at the inferential bridge such that the specific implementation of the artificial intelligence and machine learning 1910 applications can remain unmodified. This approach yields more equitable insights (e.g., accounting for equitable insights associated with a diverse patient population), boosts generalizability, and because randomized or synthetic re-weighting of machine learning models is protective, the approach improves confidentiality and privacy while optimizing utility. Further detail regarding the BIS 1924, the RLB 1924a, and the ALB 1924b is provided in relation to the following Figures.

The RLB 1924a can include modifying a number of records from a dataset from either a majority outcome (e.g., majority of a particular demographic) or a minority outcome to achieve a target ratio of majority to minority outcomes. The modification can include removing existing data records, duplicating existing data records, and generating synthetic data records. The ALB 1924b includes re-weighting of majority and minority classes (e.g., model classes associated to a majority or minority demographic). In some cases, the ALB 1924b is more targeted than the RLB 1924a because it is embedded into a derivation of insights (e.g., an output of a classification model), which is unique to the inferential bridging process.

The RLB 1924a first includes identifying a class imbalance in training data. In some cases, the distributions identified by the DCM 1920 reveal a class imbalance in the training data. The RLB 1924a includes choosing a record-level rebalancing technique, which can include oversampling, under sampling, or a combination of both. After choosing a technique, a system can then train, within the inferential bridge, a machine learning model on the rebalanced training data and evaluate performance metrics of the trained machine learning model. In some cases, the trained machine learning model is evaluated using a validation or test dataset. If performance metrics of the trained machine learning model do not meet a pre-determined threshold, the RLB 1924a can adjust the rebalancing technique or use a different technique to improve the performance of the trained machine learning model.

The ALB 1924b can be implemented with similar steps described with respect to the RLB 1924a. Instead of the record-level rebalancing techniques, the ALB 1924b includes choosing and implementing an algorithm-level rebalancing technique that can include adjusting class weights of a machine learning model or using ensemble methods. Class weighting includes assigning higher weights to a minority class and lower weights to a majority class. Ensemble methods include combining multiple classifiers to improve performance on imbalanced datasets. Combined sampling includes modifying a cost function used for training the machine learning model to prioritize correctly classifying instances from the minority class.

After execution of processes (e.g., risk evaluation, re-balancing, among others) of the modeling stage 1906, the outputs and post-processing stage 1908 processes data received from the modeling stage 1906 (e.g., randomized and balanced data). The outputs and post-processing stage 1908 includes a variable threshold optimization (VTO 1926) process and an AIML model improvement (AMI 1928) process.

The VTO 1926 determines a tradeoff metric between risk and utility with independent or coupled thresholds. The risk is associated with risk of identifying particular individuals represented in the data received from the modeling stage 1906, or other types of risk (e.g., risk related to revealing confidential data). The utility is associated with a usefulness of the data received from the modeling stage 1906 (e.g., provides accurate results and represents a true distribution of the data included in the information and data assets 1902).

In some implementations, the VTO 1926 performs attribute or record thresholding (e.g., evaluating risk of attributes or records) after the EMR 1922 calculates risk metrics. For example, the EMR 1922 can include dropping PCA components associated with a high (or alternative, a low) risk metric. In some implementations, the VTO 1926 can perform the thresholding before risk calculation based on external knowledge, pre-existing classifications, or known calibration error.

The VTO 1926 can sequence data transformations by risk ranking (e.g., transform data until a utility target is met) or by utility ranking (e.g., transform data until a risk target is met). In some implementations, the VTO 1926 determines “watershed” operating points (e.g., combination of risk and utility), in which acceptable risk metrics coincide with sufficient utility (e.g., as defined by pre-determined threshold). In some implementations, the VTO 1926 receives user input to supports user-steerable choices in sub-optimal regions. The user input can support further optimization of risk and utility outside of what is capable of algorithmic decision making performed by the VTO 1926. Further detail regarding the VTO 1926 is provided in relation to the following Figures.

In some cases, the VTO 1926 evaluates risk metrics associated with each record, one record at a time. Similarly, in some cases, the VTO 1926 evaluates risk metrics associated with each attribute, one attribute at a time.

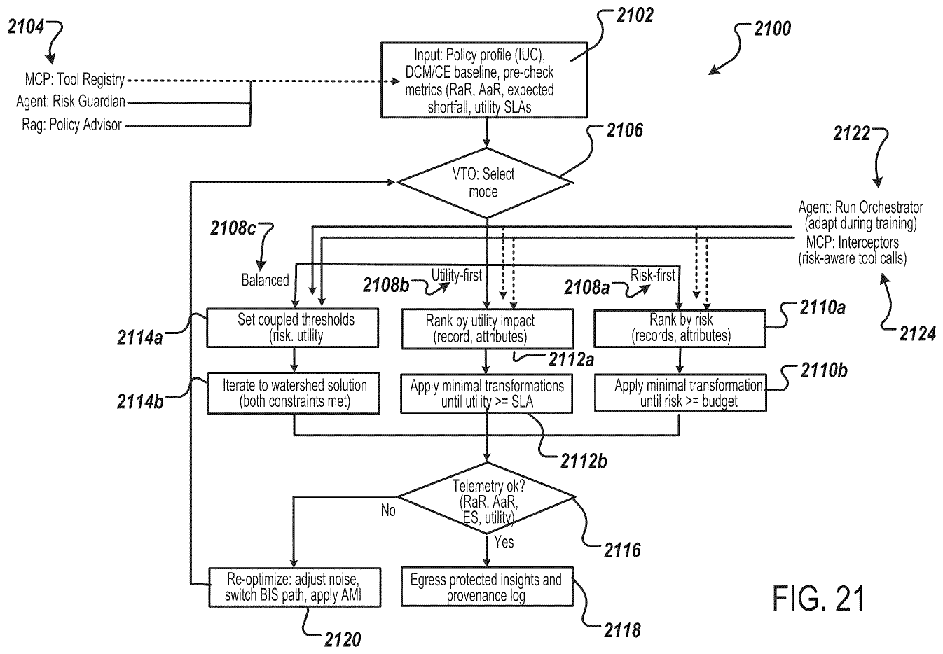

The AMI 1928 includes data processing operations like an introduction of calibrated jitter and shrinkage, differential penalization, and other inferential-bridging-aware adjustments to reduce overfitting while maintaining accuracy of the data. Because protections are placed before models process the data rather than rewriting the models, any standard AIML library can be used and still yield truthful, protected outputs. For example, regardless of particular applications included in the artificial intelligence and machine learning applications 1910, outputs of the applications 1910 are protected and yield truthful outputs due to the data process steps included in the stages 1904-1908. Further detail regarding the AMI 1928 is provided in relation to FIG. 21.

Data is transmitted from the outputs and post-processing stage 1908 to the artificial intelligence and machine learning applications 1910 via an insight support coordination (ISC 1912) process. The ISC 1912 acts as a tempo manager for rapid analytic delivery, akin to a “fire support coordination measure” for insights. Functionality of the ISC 1912 includes prioritizing data processing pipelines and marshaling correct safeguards for a particular analytical context. In some implementations, the ISC 1912 is an interface between the information and data assets 1902 and an end user that is implementing the applications 1910. The ISC 1912 manages data flow from the stages 1904-1908.

The operations associated with the stages 1904-1908 are managed and monitored by an information, risk, and utility coordination (IRUC 1914) process. The IRUC 1914 process is responsible for orchestrating distributional summaries, implementing truthfulness constraints (e.g., according to a governance protocol), and generating a suite of metrics and thresholds to steer insight discovery and data transformations according to the governance and privacy protocols. Functionality of the DSC 1916, the IRUC 1914, and the ISC 1912 can be implemented by one or more AI agents responsible for processing data in and out of the stages 1904-1908 and monitoring and configuring the processes within the stages 1904-1908.

The system implements risk-utility telemetry and decisioning. The risk-utility telemetry and decisioning includes calculating RaR, AaR, and expected shortfall, via the EMR 1922 to summarize potential losses in confidentiality and insight value over a specified time interval with corresponding confidence levels. The VTO 1926 determines an optimum transformation, based on the evaluated risk metrics, that meets risk budgets (e.g., an amount of tolerable risk, as defined by a privacy or governance protocol). The determined transformation also preserves analysis truthfulness. If thresholds are not met, the system adjusts one or more parameters (e.g., degree of randomization and cohort balancing).

The system can evaluate key performance indicators (KPIs) during processes including the IRUC 1914, the ISC 1912, and the DSC 1916. For example, the system can evaluate risk metrics (e.g., RaR and AaR at specified quantiles, expected shortfall ceilings, and per-cohort risk exposure), utility metrics (e.g., model accuracy, calibrated confidence intervals, generation gap (before and after AMI and BIS processes), and fairness differences after record and/or attribute balancing), and truthfulness records (e.g., confidence intervals at data egress). The system also evaluates and stores provenance metrics related to MCP tool calls, RAG data sources, VTO decisions, and gating outcomes.

The system provides access to various functionality according to data access modes. For example, the system can determine a particular user only accesses data via synthetic views (e.g., synthetic data) for setting up machine learning pipelines, pseudonymized views inside a secure data enclave (e.g., view-only access with no access to data extracts), or containerized and federated data access via inferential components (e.g., access to insights, rather than raw data).

FIG. 20 illustrates an example system 2000 that includes a control plane 2004, a user plane 2002, and a data plane 2006. The control plane 2004 and the user plane 2002 are examples of the control plane 1706b and the user plane 1706a respectively of the inferential bridge 1706 described in relation to FIG. 17. The data plane 2006 is an example of the data plane 1704 as described in relation to FIG. 17. The example system 2000 is an example implementation of the example process 1900 described in relation to FIG. 19.

The example system 2000 includes access points for AI agents to perform activities including retrieval-augmented generation (RAG) functionality and accessing data associated with a model context protocol (MCP) to access tools and external resources.

A user, e.g., a developer or analyst, executes functions by interacting with a user interface that operates within the user plane 2002. For example, the user can execute (2002a) code or submit (2002b ) a job to be executed by a remote processor on secure and confidentialized data via interaction with a virtual notebook (e.g., a Jupyter Notebook), SQL, or a machine learning development framework. The code and processes executed in the user plane 2002 can be unmonitored and unmodified. Data that the code processes are first processed by a system that implements the example process 1900, e.g., inferential bridging.

Upon submitting (2002b ) a job, a control plane 2004 receives a trigger indicative of the submitted job or indicative of a scheduled data processing pipeline. For example, the user can submit a job that includes training a machine learning model using protected data that is stored in resource located in a data plane 2006. The job can include an instruction similar to “Train a rare-disease regression model”, in which the regression model is trained on a labeled rare-disease dataset with a large class imbalance of 2% of data principals associated with a rare disease. The control plane 2004 pulls relevant data from the data plane 2006 according to a determined access mode and processes the relevant data according to the submitted job from the user plane 2002.

An IRUC 2008 loads a relevant policy profile associated with the user interacting with the user plane 2002, data involved in the submitted job, and details related to the submitted job. The relevant policy profile can include risk budgets (e.g., an amount of tolerable risk). The risk budgets can include metrics like RaR and AaR quantiles, expected shortfall, and utility agreements. The relevant policy profile can also include truthfulness constraints (e.g., valid intervals and other utility metrics).

In some implementations, a policy advisor AI agent 2010a can perform RAG operations that include processing the relevant policy profile loaded by the IRUC 2008 as well as governance playbooks, risk catalogs, threshold catalogs, and cohort equity guidance for the project context (e.g., context of the submitted job), and other authoritative references. The policy advisor AI agent 2010a can process the loaded data with a large language model (LLM) to generate an output indicative of outputs associated with the IRUC 2008 (e.g., distributional properties, truthfulness constraints, and other metrics and thresholds associated with steering insight discovery and data transformation). In some cases, the system 2000 advertises safe and allowed tools (e.g., to interact with external APIs) and data scopes accessible to the AI agent 2010a via MCP-style capabilities. The policy advisor AI agent 2010a is configured to generate human-readable explanations of thresholds selected by the IRUC 2008.

Based on the relevant policy profile and the submitted job, a DSC 2012 determines an access mode for retrieving data from the data plane 2006. The access mode can allow the user to access data from a synthetic foundry 2006a (e.g., for machine learning pipeline setup), a pseudonymized enclave 2006b (e.g., for preparation and data manipulation without access to model outputs), or a federated and containerized data source 2006c (e.g., for accessing inferential outputs across an inferential bridge). Different tasks (e.g., different types of submitted jobs) require different access modes. The DSC 2012 facilitates retrieval of data according to the determined access mode from the data plane 2006 to the control plane 2004. In some cases, the DSC 2012 first provides data from the pseudonymized enclave 2006b for data feature preparation then the DSC 2012 retrieves data from the federated and containerized data source 2006c for the inferential bridge to provide inferential components (e.g., sufficient statistics or information matrices for insight extraction) to the user.

Upon receiving the data from the data plane 2006 at the control plane 2004, the system 2000 implements a DCM and CE 2014 process. The DCM process includes capturing distributions of the data (e.g., distributional properties) and checking for drift of the distributions against prior baselines. For the example of the logistic regression, the DCM process includes determining a full feature space of the data and a distribution of labels (e.g., rare, not rare, etc.) and identifying a significant label imbalance (e.g., 2% of the labels are indicative of a rare disease).

Modeling of the CE facilitates modeling of implicit, inferred, and verification errors in the data to refine risk and utility metric estimates. For the example of the logistic regression, the CE is indicative of potential verification error (e.g., label noise in rare outcomes). In some implementations, the DCM and CE 2014 process are implemented by a data sentry AI agent 2010b configured to summarize outputs of the DCM and CE 2014 process and to propose initial parameters for an EMR process to minimize an amount of noise added to the data.

Upon the DCM and CE 2014 process outputting the distributional properties of the data and correcting for calibration error, a risk checker 2016 implements a risk pre-check that includes a calculation of RaR, AaR, and expected shortfall of the data. In some implementations, a risk guardian AI agent 2010c compares the calculated risk metrics against a risk budget associated with the submitted job and flags hotspots that indicate elevated risk (e.g., particular records, attributes, and cohorts). In some cases, the risk guardian AI agent 2010c can determine if a driving factor for elevated risk can be associated with class imbalance (e.g., a large majority class). If this is the case, the agent 2010c can pre-plan BIS intervention via RLB and/or ALB. For the example of the logistic regression, the agent 2010c can determine combinations of attributes with the rare outcome label that could lead to high-risk inferences for a small sub-cohort of data principals. Due to the imbalance of labels in the rare-disease dataset (e.g., 2% of data principals associated with a rare disease), the risk guardian AI agent 2010c can schedule a BIS process to be implemented downstream, as discussed below.

A transformation planner 2018 executes a VTO process and an EMR process. The VTO process includes choosing an optimization strategy. Example optimization strategies include a risk-first strategy (e.g., fixed risk and maximize utility), a utility-first strategy (e.g., fixed utility and minimize risk), or a balanced strategy (e.g., Pareto). The transformation planner 2018 can determine a sequence of transformations at the attribute and/or record level based on risk and/or utility rankings. The EMR process can apply a PCA or cluster weighting process to reduce the size of a solution space and to target added noise and/or jitter on data values or data value ranges that exhibit high variance and/or high risk. In some implementations, a utility optimizer AI agent 2010d implements functionality of the transformation planner 2018 by executing candidate optimization strategies, simulating metric differences between different strategies, recommending minimal data transformations, and providing text-based and human-readable explanations of risk-utility trade-offs for IRUC approval (e.g., against risk budgets).

A modeling loop 2020 includes an AMI process and a BIS process. The AMI process is implemented if the submitted job requires tuning a machine learning model (e.g., the logistic regression model). The BIS process is implemented if the submitted job requires processing dat with a record or attribute imbalance.

In a case in which the user submits code to be executed from the user plane 2002, the modeling loop 2020 executes the submitted code unchanged. The AMI process includes applying calibrated data shrinkage and/or penalties of jitter at safe time hooks (e.g., time points during model training or validation) to avoid overfitting while preserving accuracy of the model outputs and truthful statistics. In a case in which the modeling loop 2020 receives data from the transformation planner 2018, the modeling loop 2020 also executes code associated with the submitted job from the user unchanged.

If record or attribute balancing is required, the modeling loop 2020 implements an application of RLB (e.g., oversampling, under sampling, or generation of synthetic data) and/or ALB (e.g., class weights, ensembles, or cost-sensitive parameters). For the example of the logistic regression, RLB is applied to either include synthetic examples of rare disease labels or under sampling the set of non-rare disease labels in the dataset. In some implementations, a run orchestrator AI agent 2010e manages guardrails associated with a risk policy and documents every decision made by each AI agent of the system 2000 implemented within the control plane 2004 in a data storage device.

The run orchestrator AI agent 2010e can implement real-time risk telemetry and adaptation by periodically (e.g., after each data transformation) the RaR, AaR, and Expected shortfall on intermediate results. If risk thresholds drift, then the agent 2010e can initiate the VTO to re-optimize the transformation strategy (e.g., switch from ALB to RLB) or modify parameters of the chosen strategy (e.g., increase noise). The run orchestrator AI agent 2010e coordinates callbacks during machine learning training and risk modeling. The agent 2010e can also switch BIS, EMR, and AMI strategies according to the real-time risk telemetry outputs.

In some implementations, an MCP interceptor AI agent 2010f manages tools accessible to AI agents of the system 2000 (e.g., model trainers, model evaluators, feature stores, etc.). The MCP interceptor AI agent 2010f can process policies to determine which actions and which tools are accessible to processors and agents at different stages of data processing within the control plane 2004. The MCP interceptor AI agent 2010f can determine access scopes and rate limits associated with particular access modes. Furthermore, the MCP interceptor AI agent 2010f performs risk-aware actions such that each implementation of an external tool (e.g., pull data, train model, export data) first passes through the MCP interceptor AI agent 2010f to determine if an intervening process is required to ensure risk and utility metrics meet appropriate thresholds. Data associated with accessing external tools by AI agents can be included in provenance logs for future analysis and processing.

The described control-plane safeguards can be deployed within a TRE/SPE so that all data touchpoints and tool invocations occur inside a governed enclave with dual egress approval. An egress decision process 2022 is implemented to validate risk budgets defined in the risk protocol relative to evaluated risk by the run orchestrator agent 2010e and utility agreements. The egress decision process 2022 transmits, upon risk and utility metrics being met, protected insights (e.g., outputs of a trained machine learning model, model coefficients, standard errors, valid confidence intervals, calibrated performance metrics, and safe aggregates). The egress decision process 2022 can include egress gates that produce provenance logs 2024. The provenance logs 2024 can include distributional properties observed in the data, transformations applied, choices related to the BIS, EMR, and AMI, VTO rationale, and risk and utility metrics and thresholds that were satisfied. In some implementations, a reporting AI agent 2010g can process the provenance log using a RAG pipeline to generate human-readable reports by accessing metric glossaries, risk policy snippets, etc., and linking to evidence provided by the provenance log.

A post-run learning process 2026 refreshes DCM baselines (e.g., re-evaluates empirical distributional properties of the data), updates CE models, generates risk portfolio dashboards (e.g., at a project or program level) and generates data for oversight by the IRUC, ISC, and DSC processes. In some implementations, a trend sentinel AI agent 2010h monitors outputs of the post-run learning process 2026 to identify systematic imbalance or repeated near-misses on risk budgets and is configured to propose risk policy or risk profile updates for future jobs. For the example of the logistic regression, a generated output by the post-run learning process 2026 can be similar to

```
{
  ″model″: ″logistic_regression″,
  ″positive_class_prevalence″: 0.021,
    ″BIS″: {
      ″RLB″: ″synthetic minority generation (shadowed inside bridge)″,
      ″ALB″: ″class_weight=pos:10.5, neg:1.0″
  },
      ″AMI″: ″calibrated shrinkage on high-variance coefficients″,
  ″coefficients″: [
      {
        ″feature″:″age″,
        ″beta″:0.042,
        ″se″:0.010,
        ″ci95″:[0.022,0.062]
      },
      {
        ″feature″:″biomarker_A″,
        ″beta″:1.37,
        ″se″:0.21,
        ″ci95″: [0.96,1.78]
      }
  ],
      ″metrics″: {
      ″AUC_val″: 0.83,
      ″ECE″: 0.018
  },
  ″risk_metrics″: {
      ″RaR@q=0.99″: 0.021,
      ″AaR@q=0.99″: 0.017,
      ″ExpectedShortfall@q=0.99″: 0.028
  },
      ″egress″: ″parameters, intervals, safe aggregates only; no row-level data”
}
```

The trend sentinel AI agent 2010h can process various corpora within the control plane as part of the RAG pipeline. The corpora can include internal policy manuals, threshold catalogs, standardized documentation related to machine learning models, data dictionaries, provenance logs, cohort definitions, and approval playbooks. Each output of the RAG pipeline that leads to a decision (e.g., updated risk profile) includes citations indicative of reasoning behind the decision. The RAG pipeline is configured to process data associated with processes executed by the system 2000 and does not include processing of raw protected data (e.g., data stored in the data plane 2006).

The example system 2000 can be configured to operate according to one or multiple operating modes. For example, the system 2000 can operate in a non-interactive batch mode. The non-interactive batch mode includes a single pass from submitting a job to generating a model output with a single optimization strategy. The non-interactive batch mode does not include modified optimization strategies, and is a preferred mode for reproducible studies and scheduled jobs. The non-interactive batch mode typically implements a conservative VTO and emphasizes provenance completeness, reproducibility, and truthfulness of data intervals.

As another example, the system 2000 can operate in an interactive (e.g., research) mode. The interactive mode includes a feedback loop between the DCM and CE 2014 process and the generation of the model output. A preferable data access mode is a synthetic foundry 2006a or the pseudonymized enclave 2006b for data exploration. The data access mode can switch to the federated and containerized data source 2006c for a final run after the feedback loop is complete and target thresholds are met. The interactive mode emphasizes developer velocity with safe guardrails with minimal required refactoring.

As another example, the system 2000 can operate in a federated multi-party mode. The federated multi-party mode includes local storage of data, in which the system 2000 only passes inferential components (e.g., information matrices) to an end user. Processes executed by the system 2000 including the BIS process, the EMR process, and the VTO process aggregated risk metrics without determining risk metrics on a centralized set of data records. The federated multi-party mode emphasizes data sovereignty and negotiated risk budgets across parties.

As another example, the system 2000 can operate in a streaming (e.g., digital twin) mode. The streaming mode includes executions of the full set of operations described in relation to the system 2000 continuously. The system 2000 monitors risk metric drifts and modifies calibration error as needed. The system 2000 implements the VTO to throttle data transformations in an event in which parameters should be modified. The streaming mode can enable real-time (e.g., fast) access to protected data. The streaming mode emphasizes stability under metric drift and real-time insight protection.

As another example, the system 2000 can operate in a high-assurance regulatory mode that can include strict risk budgets, dual egress approval, strong expected shortfall ceilings, and expanded provenance requirements. The high-assurance regulatory mode emphasizes auditable compliance with truthful guarantees of outputs.

FIG. 21 illustrates a representation of an example process 2100 for determining a data optimization strategy as part of a VTO (e.g., the VTO 1926 described in relation to FIG. 19) process. The example process 2100 is implemented by a system that is configured to execute instructions associated with an inferential bridge that is positioned between protected data sources and end users that receive protected insights (e.g., outputs of machine learning models).

The system receives input data 2102. The input data includes a policy profile, DCM outputs, CE baselines, pre-check risk metrics including RaR, AaR, expected shortfall, and utility service agreements. The system receives the input data 2102 from multiple data sources 2104 including an MCP tool registry, a risk guardian AI agent, and a RAG policy advisor, as described above.

Based on the input data 2102, the system selects (2106) a data optimization strategy. The data optimization strategies include a risk-first strategy 2108a (e.g., fixed risk and maximize utility), a utility-first strategy 2108b (e.g., fixed utility and minimize risk), or a balanced strategy 2108c (e.g., Pareto).

The risk-first strategy 2108a includes ranking (2110a) records or attributes by risk and applying (2110b) minimal data transformations until a measured risk is greater than or equal to a risk budget, as defined in the input data 2102. The utility-first strategy 2108b includes ranking (2012a) the records or attributes by utility and applying (2012b) minimal data transformations until a measured utility is greater than or equal to a service agreement, as defined in the input data 2102. The balanced strategy 2108c includes setting (2114a) coupled thresholds for utility and risk and iterating (2114b) an optimization process until a watershed solution is found (e.g., both thresholds are met).

The system determines (2116) if relevant threshold are met (depending on which strategy is chosen). If the relevant thresholds are met, the system proceeds to egress (2118) protected insights (e.g., machine learning outputs) to an end user and to store all activity performed as part of the example process 2100 in provenance logs for future analysis. If the relevant thresholds are not met, the system re-optimizes (2120) parameters of the data transformation (e.g., more or less noise, modified BIS path, or modified AMI process). Upon re-optimization, the system selects (2106) a data optimization strategy.

In some implementations, AI agents perform one or more tasks related to the example process 2100. In some implementations, an orchestrator AI agent 2122 evaluates risk and applies noise to data attributes or records for each data optimization strategy. In some implementations, an MCP interceptor AI agent 2124 manages interaction between AI agents (e.g., the orchestrator AI agent 2122) and external resources based on calculated risk.

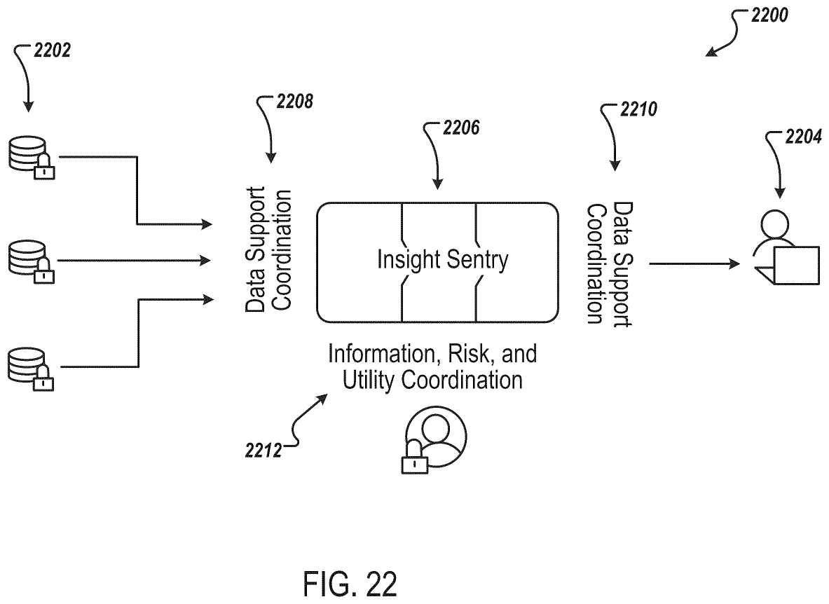

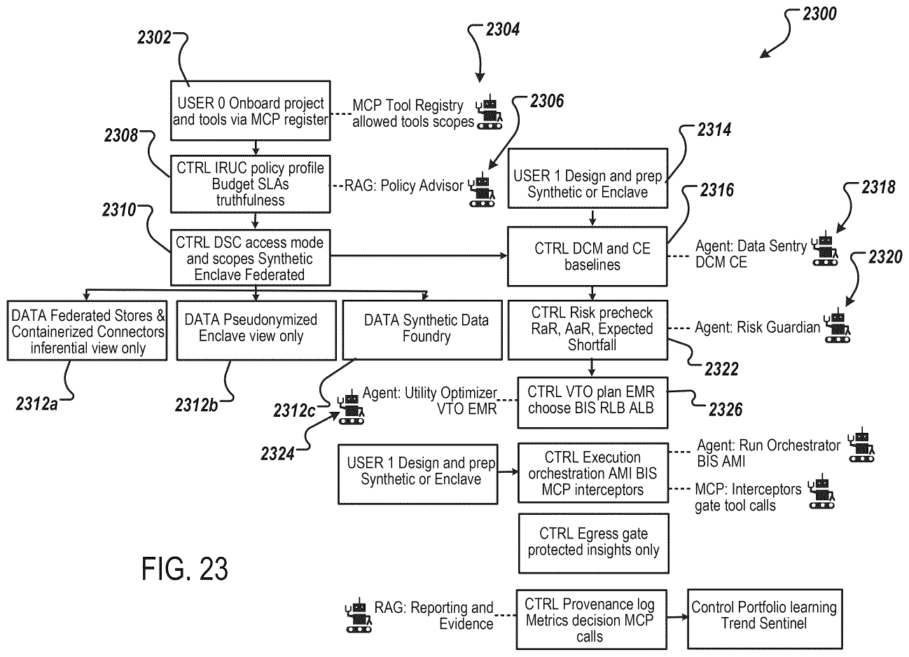

FIGS. 19-21 describe systems and processes related to internal functionality of an inferential bridge, in which protected data are transformed into protected insights. The system related to FIGS. 19-21 can be referred to as an “insight sentry” or an “insight watchtower,” in which the system includes processes for monitoring and transforming data across the inferential bridge. FIGS. 22-23, which are described below, relate to a platform level description of systems and processes related to the inferential bridge. The systems related to FIGS. 22-23 can be referred to as an “insight command” that orchestrates functionality of the insight sentry, e.g., providing configuration parameters to the insight sentry. The insight command includes governance user consoles, tool gating for AI agents (e.g., via MCP), access-mode orchestration (e.g., restricting access to particular users), and portfolio-level provenance (e.g., combined provenance for a group of tasks or datasets).

FIG. 22 illustrates an example system 2200 for implementing functionality of an insight command. The insight command is a system-of-systems configured to orchestrate data processing tasks across a user plane (e.g., the user plane 2002), a control plane (e.g., the control plane 2004), and a data plane (e.g., the data plane 2006), as described in relation to the example system 2000 of FIG. 20.

The insight command is configured to register data and risk policies (e.g., uploaded by users or data administrators), to publish allowed AI agent tools via an MCP tool registry, to determine access modes via DSC, to provision data workspaces according to synthetic, pseudonymized, and federated data access modes, and to coordinate provenance and portfolio dashboards.

The insight sentry (as described in relation to FIGS. 19-21) is configured to implement data processing tasks, e.g., DCM and CE processes, risk evaluation, VTO, EMR, BIS, and AMI. Implementation of the insight sentry ensures that truthful and protected insights exit the inferential bridge while allowing unmodified user code to be executed.

The system 2200 facilitates the transformation of datasets 2202 into protected insights to be received by a user 2204 by an insight sentry 2206, with functionality similar to the insight sentry described above. The processes within the insight sentry 2206 are managed via IRUC process 2212, as described in relation to FIG. 20. In some implementations, the IRUC process 2212 implements needs-based information governance management providing access and determining risk tolerances based on specific needs of the user 2204 and characteristics of datasets 2202.

The datasets 2202 can be federated across secure data stores and can be used to produce synthetic versions that users can view (e.g., via a synthetic foundry) for establishing AI and ML pipelines. The datasets 2202 are ingested into the insight sentry 2206 via a DSC process 2208, as described in relation to FIG. 20.

The user 2204 can access the protected insights using various interfaces (e.g., Jupyter Notebooks or any other software framework) with automated protection of insights that are drawn from the datasets 2202. The protected insights are delivered to the user 2204 via an ISC process 2210, as described in relation to FIG. 20.

FIG. 23 illustrates an example process 2300 that represents functionality of an insight command system that manages operations of the insight sentry, as described in relation to FIG. 20. The example process 2300 can be executed by a computing system that is communicatively coupled to processes that are configured to execute data processing tasks in each of three planes including a user plane, a data plane, and a control plane.

A user onboards (2302) a project and relevant tools to be accessed by AI agents via an MCP registry. In the context of the example use case related to the rare-disease logistic regression, the user onboards (2302) a project “Rare-Disease Regression” and attaches a policy profile to the project. The policy profile can include risk budgets (e.g., RaR/AaR at q=0.99 (quartile), and expected shortfall ceiling), truthfulness constraints (e.g., regression confidence intervals must remain valid at egress), utility agreements (e.g., calibration error less than or equal to 0.02), and a tool-allow list via the MCP tool registry (e.g., scikit-learn logistic Python package, and approved model evaluators). In some implementations, an MCP tool registry AI agent 2304 facilitates extraction of data from the MCP tool registry.

A RAG policy advisor AI agent 2306 retrieves (2308) approved policy playbooks, threshold catalogs, and equity and balancing guidelines. The retrieved documentation is associated with citations for the RAG policy advisor AI agent 2306 to provide auditability.

The system performs a DSC process to determine (2310) a data access mode. The DSC process includes declaring default access modes and fallback modes. The system also determines MCP scopes (e.g., which connectors and external functions are available to the user and to AI agents).

The system configures a data workspace according to the determined data access mode. The system can configure a synthetic foundry workspace 2312a for feature engineering and pipeline scaffolding development, a pseudonymized enclave workspace 2312b for view-only data access for hands-on data preparation, and a federated and containerized data workspace 2312c to pass inferential components across the inferential bridge.

The user executes (2314) computer code associated with the project (e.g., via a digital notebook, SQL, machine learning package, etc.). In some implementations, the execution includes designing and preparing a dataset from a synthetic foundry or a pseudonymized enclave. Upon code execution, the system (e.g., within the insight sentry) performs (2316) DCM and CE processes. In some implementations, the DCM and CE processes are performed by a data sentry DCM CE AI agent 2318.

A risk guardian AI agent 2320 implements (2322) a risk pre-check process. The agent 2320 computes initial values for RaR, AaR, and expected shortfall and compares the initial values against project budgets loaded in the policy profile. A VTO process, e.g., via an optimizer AI agent 2324, includes selecting (2326) an optimization strategy. For example, the agent 2324 can select a balanced mode to meet risk and utility budgets simultaneously. The agent 2324 can also propose a particular BIS path to account for imbalance in the data (e.g., 2% rare disease labels in the example dataset described above), and EMR weighting strategy (e.g., PCA or clustering). The agent 2324 can also simulate candidate optimization plans, generate human-readable trade-off explanations, and propose a minimal transformation plan that satisfy risk budgets and generate maximal utility.

The system, and in some cases, an AI agent, executes processes subsequent to the selecting of the optimization strategy, as described in relation to FIG. 20 (e.g., execution loop, egress decision making, and post-run learning).

Execution of the example process 2300 can be initiated and configured by an external user via an operational runbook. For example, a YAML-formatted runbook for executing the example logistic regression job related to classification of rare diseases described above can be written as
project: Rare-Disease Regressionplanes:
   user: Jupyter/ML framework (no algorithm rewrites)
   control: Insight Sentry+IRUC/ISC/DSC+event/log bus
   data: federated stores+synthetic foundry+pseudonymized enclave
onboarding:
   IRUC.policy_profile:
      risk_budgets: {RaR_q: 0.99, AaR_q: 0.99, ES_max: “<ceiling>”}
      truthfulness: “valid coefficient CIs at egress”
      utility_SLA: {AUC_min: 0.80, ECE_max: 0.02}
   MCP.tool_registry:
      allow: [“scikit-logistic”, “xgboost”, “approved-evaluators”]
   RAG.policy_advisor: “attach policy citations”
data_registration_and_modes:
   DSC:
      default_ladder: [“synthetic”, “enclave”, “federated”]
      inferential_components_allowed: [“information_matrix”, “safe_aggregates”]
   MCP.scopes: “publish connector/action capabilities to runtime”
workspace_provisioning:
   synthetic_foundry: “design-time shadow for feature engineering”
   enclave: “view-only prep; no extracts”
   federated_connectors: “inferential-only for execution”
pre_run_checks:
   Sentry. DCM_CE_baseline: “profile label & features; quantify CE”
   Sentry. RiskGuardian.snapshots: [“RaR”, “AaR”, “ExpectedShortfall”]
   hotspots: “tiny cohorts combining rare outcome+high-risk features”
optimization_planning:
   VTO.mode: “balanced” BIS:
      RLB: “minority oversampling/synthetic (inside bridge)”
      ALB: “class weights/cost-sensitive loss (e.g., pos_weight≈10.5)”
   EMR: “PCA/cluster weighting to focus noise where variance/risk concentrate”
   UtilityOptimizer: “simulate plans; recommend minimal-change plan”
status: “Ready to enter execution loop”

The example YAML-formatted runbook includes information required by the insight sentry and the insight command to implement the training of the logistic regression model and the inferential bridge to provide protected insights based on the model trained on protected data.

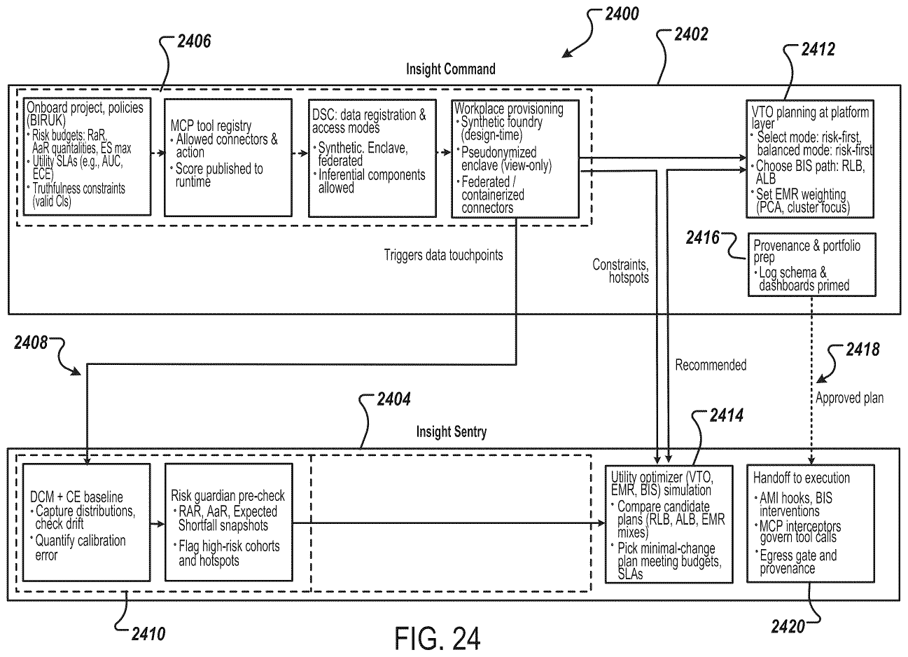

FIG. 24 illustrates an example system 2400 that includes an insight command 2402 and an insight sentry 2404. The insight command 2402 executes a process similar to the example process 2300 described in relation to FIG. 23. The insight sentry 2404 is similar to the example system 2000 described in relation to FIG. 20. The example system 2400 includes methods of interaction between the insight command 2402 and the insight sentry 2404.

The insight command 2402 implements functionality 2406 described in relation to FIG. 23. For example, the insight command 2402 onboards a project, sets risk budgets, sets utility agreements, determines truthfulness constraints. Furthermore, the insight command 2402 registers tools accessible to AI agents via an MCP tool registry, determines a data access mode, and provisions a data workspace according to the determined data access mode.

Upon executing the functionality 2406, the insight command 2402 transmits a data touchpoint trigger 2408 to the insight sentry 2404. Upon receiving the data touchpoint trigger 2408, the insight sentry 2404 performs functionality 2410 that includes DCM and CE baseline processes and a risk pre-check (e.g., determining RaR, AaR, expected shortfall, and flagging high-risk cohorts and attributes).

Upon executing the functionality 2406, the insight command 2404 can also perform a VTO planning process 2412 to select an initial data optimization strategy, determine a BIS path (e.g., RLB, ALB, or both), and set EMR weighting (e.g., for PCA or clustering). The insight command 2404 can perform the VTO planning process 2412 subsequent to or in parallel to transmitting the data touchpoint trigger 2408 to the insight sentry 2404.

The insight sentry 2404 implements a VTO process 2414 upon receiving constraints as a result of the execution of the functionality 2406 and upon receiving the distributional properties and risk metrics as a result of the execution of the functionality 2410. The VTO process 2414 includes performing simulations to compare candidate plans, based on the initial plan generated by the VTO planning process 2412. The VTO process 2414 also includes determining a minimal-transformation plan that meets risk budgets and utility agreements.

The insight command 2402 implements a provenance and portfolio preparation process 2416 to initialize log schemas and dashboards. The insight sentry 2404 can receive an approved plan 2418 from the provenance and portfolio preparation process 2416 to execute a remaining process 2420 implemented by the insight sentry 2404 that includes an AMI process, BIS interventions, MCP interceptors to govern interaction with external tools by AI agents, and egress gate management.

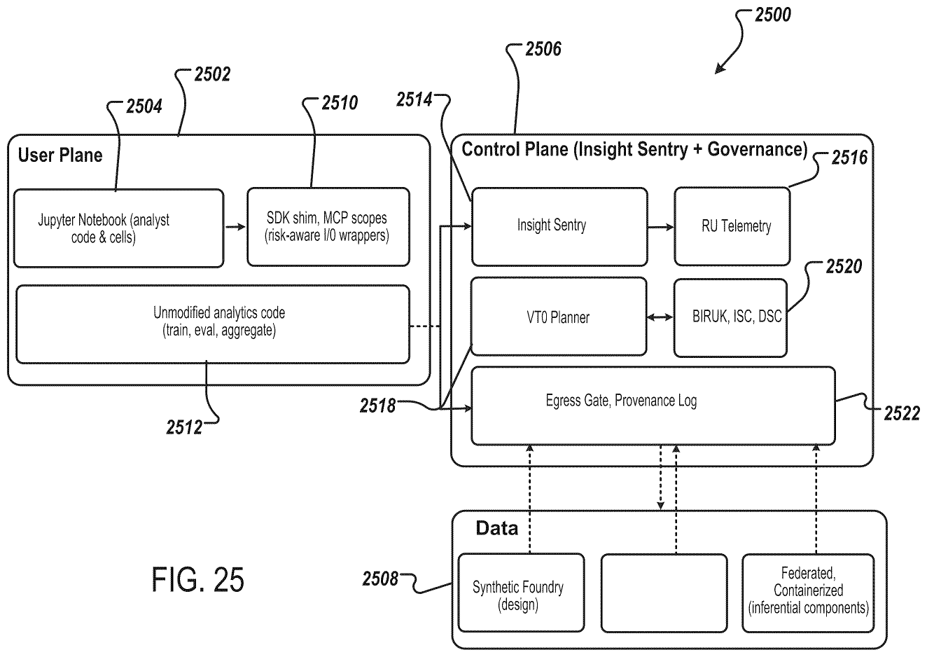

FIG. 25 illustrates an example system 2500 that includes a user interface 2504 in a user plane 2502 communicatively coupled to a control plane 2506. The control plane 2506 is communicatively coupled to a data plane 2508 and mediates access by a user operating within the user plane 2502 to data stored and managed in the data plane 2508 via inferential bridging. The data plane 2508 includes a synthetic foundry, a pseudonymized enclave, and a federated and containerized data configuration, as described in relation to the previous Figures.

The control plane 2506 implements an insight sentry 2514, as described in relation to FIG. 20, a VTO planning process 2518, as described in relation to the insight command 2402 of FIG. 24, a risk-utility telemetry process 2516 to monitor the trade-off between risk and utility of a transformed dataset, and IRUC, ISC, and DSC processes 2520 for managing privacy and governance protocols and setting thresholds, delivering protected insights, and ingesting data respectively.

The control plane 2506 also implements an egress gate and a provenance log 2522 to determine which insights can leave the control plane 2506 and to log all actions taken by processors and agents operating within the control plane 2506.

The user plane 2502 includes the user interface 2504 in the form of a Jupyter notebook. An analyst can interact with the Jupyter notebook to write and execute computer code related to a particular job (e.g., training a logistic regression model on labeled rare-disease data). The user interface 2504 can interact with a software development kit (SDK) to access MCP scopes (e.g., access protocols for external tools). The user interface 2504 can trigger an execution of code written by the analyst. Upon triggering the execution of the code, the control plane 2506 initiates the insight sentry and related processes described herein.

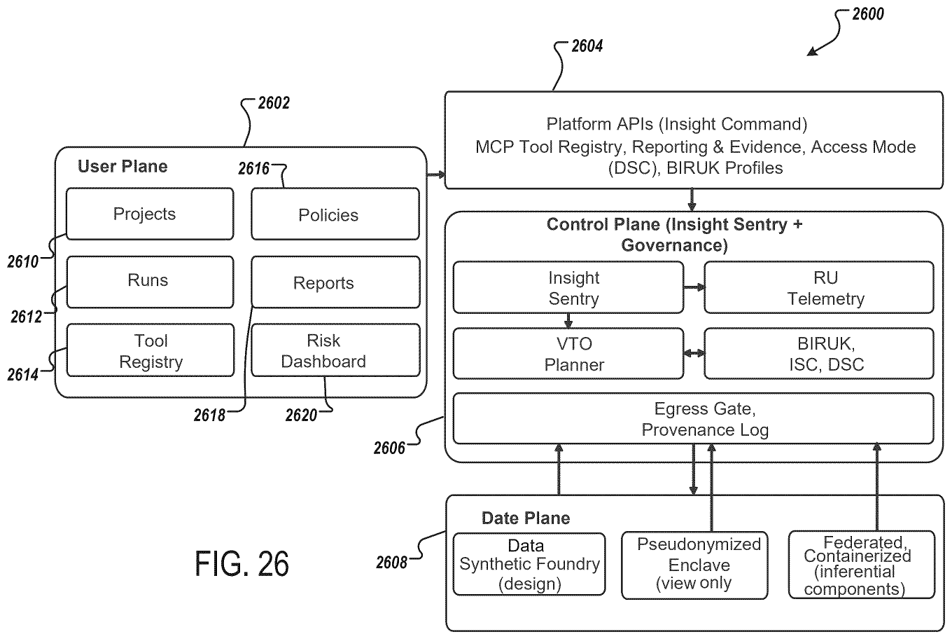

FIG. 26 illustrates an example system that includes a user interface in a user plane 2602 communicatively coupled to a control plane 2606 via a set of platform APIs 2604. The control plane 2606 is communicatively coupled to a data plane 2608. The control plane 2606 and the data plane 2608 are operationally similar to the control plane 2506 and the data plane 2508 described in relation to FIG. 25.

The platform APIs 2604 facilitate access to an MCP tool registry, reporting and evidence resources, access mode resources, and IRUC profiles, and provide an access point to the control plane 2606.

The user interface of the user plane 2602 includes a variety of data views accessible to a user, e.g., an analyst. The views include a list of initiated projects 2610, a list of runs 2612 associated with each of the projects 2610, a tool registry 2614, polices 2616 (e.g., risk policies) associated with each of the projects 2610, reports 2618 (e.g., generated by the control plane 2606 upon executing one of the projects 2610), and a risk dashboard 2620.

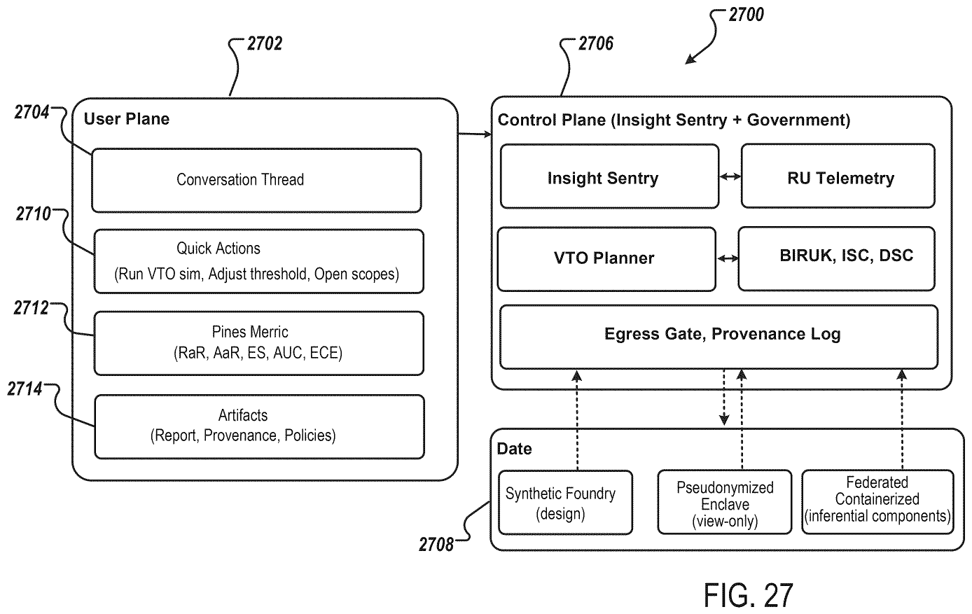

FIG. 27 illustrates an example system that includes a chat interface in a user plane 2702 communicatively coupled to a control plane 2706. The control plane 2706 is communicatively coupled to a data plane 2708. The control plane 2706 and the data plane 2708 are operationally similar to the control plane 2506 and the data plane 2508 described in relation to FIG. 25.

The chat interface includes a conversation thread 2704 between an analyst and a conversational AI agent that has access to computing and networking resources that are communicatively coupled to the control plane 2706. An interaction between the analyst and the conversational AI agent can include quick actions 2710 to initiate processes executed in the control plane 2706. For example, the quick actions 2710 can include “run VTO simulation,” “adjust thresholds,” and “open scopes.” The conversational AI agent can also provide pinned metrics 2712 to display to the analyst within the conversation thread 2704. The pinned metrics 2712 can include RaR, AaR, expected shortfall, among others. The conversational AI agent can also provide artifacts 2714 to the analyst that include reports, provenance, and policies.

Embodiments of the subject matter and the functional operations described in this specification can be implemented in digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible non-transitory program carrier for execution by, or to control the operation of, data processing apparatus. Alternatively or in addition, the program instructions can be encoded on an artificially-generated propagated signal, e.g., a machine-generated electrical, optical, or electromagnetic signal, that is generated to encode information for transmission to suitable receiver apparatus for execution by a data processing apparatus. The computer storage medium can be a machine-readable storage device, a machine-readable storage substrate, a random or serial access memory device, or a combination of one or more of them. The computer storage medium is not, however, a propagated signal.

The term “data processing apparatus” encompasses all kinds of apparatus, devices, and machines for processing data, including by way of example a programmable processor, a computer, or multiple processors or computers. The apparatus can include special purpose logic circuitry, e.g., an FPGA (field programmable gate array) or an ASIC (application-specific integrated circuit). The apparatus can also include, in addition to hardware, code that creates an execution environment for the computer program in question, e.g., code that constitutes processor firmware, a protocol stack, a database management system, an operating system, or a combination of one or more of them.

A computer program (which may also be referred to or described as a program, software, a software application, a module, a software module, a script, or code) can be written in any form of programming language, including compiled or interpreted languages, or declarative or procedural languages, and it can be deployed in any form, including as a stand-alone program or as a module, component, subroutine, or other unit suitable for use in a computing environment. A computer program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub-programs, or portions of code. A computer program can be deployed to be executed on one computer or on multiple computers that are located at one site or distributed across multiple sites and interconnected by a communication network.

Computers suitable for the execution of a computer program include, by way of example, can be based on general or special purpose microprocessors or both, or any other kind of central processing unit. Generally, a central processing unit will receive instructions and data from a read-only memory or a random access memory or both. The essential elements of a computer are a central processing unit for performing or executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

Embodiments of the subject matter described in this specification can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (“LAN”) and a wide area network (“WAN”), e.g., the Internet.

While this specification contains specific implementation details, these should not be construed as limitations on the scope of any invention or of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous.

What is claimed is

1. A method for generating a trained machine learning model trained on a plurality of segregated data sources, the method comprising:
generating, in a first segregated environment, a first dataset by transforming a first source dataset, the transforming including generating an embedded representation of the first source dataset and adding privacy parameters to the first source dataset;
generating, in a second segregated environment, a second dataset by transforming a second source dataset, the transforming including generating an embedded representation of the second source dataset and adding privacy parameters to the second source dataset;
generating a combined dataset comprising the first dataset and a ground truth dataset from the first segregated data environment combined with the second dataset from the second segregated data environment, wherein the first dataset and the ground truth dataset are stored in a first bridge database, and the second dataset is stored in a second bridge database; and
training a machine learning model with training data, the training data comprising a subset of the combined dataset, wherein model parameters of the trained machine learning model are stored in a storage device.

2. The method of claim 1, wherein the first source dataset corresponds to health data.

3. The method of claim 1, wherein the privacy parameters comprise injected noise.

4. The method of claim 1, wherein the second source dataset corresponds to consumer data.

5. The method of claim 1, wherein the transformation of the first source dataset is performed by a first artificial intelligence (AI) agent operating within the first segregated data environment, and wherein the transformation of the second source dataset is performed by a second AI agent operating within the second segregated data environment.

6. The method of claim 5, wherein the transformation of the first source dataset and the transformation of the second source dataset are each performed by processing the respective dataset with an embedding neural network model.

7. The method of claim 5, wherein the second AI agent is configured to receive the transformation of the first source dataset from the first AI agent using a model context protocol (MCP) framework of communication between AI agents.

8. The method of claim 1, wherein training the machine learning model is performed by a model training artificial intelligence (AI) agent operating within a model training environment, wherein the model training environment is different from the first segregated data environment and different from the second segregated data environment.

9. The method of claim 1, further comprising validating the trained machine learning model by a model validation artificial intelligence (AI) agent, wherein the validation comprises evaluating the trained machine learning model based on a subset of the training data, and wherein the model validation AI agent is configured to receive model parameters of the trained machine learning model.

10. The method of claim 9, wherein the validation further comprises verifying calibration of predicted probabilities.

11. The method of claim 9, further comprising transmitting, from the model validation AI agent to an entity operating within a model serving data environment, results of the validation.

12. The method of claim 9, further comprising storing the model parameters of the trained machine learning model in a storage device within a model serving data environment.

13. The method of claim 11, further comprising receiving, at a model inference AI agent operating within the model serving data environment, a task signal from the entity operating within the model serving data environment, wherein the task signal initiates a model inference process performed by the model inference AI agent.

14. The method of claim 13, further comprising loading, by the model inference AI agent, the model parameters of the trained machine learning model from the storage device within the model serving environment to perform the model inference process.

15. The method of claim 13, further comprising transmitting, from the model inference AI agent to a delivery AI agent operating within the model serving data environment, results of the inference process, wherein the delivery AI agent is configured to package the results of the inference process for consumption by a second entity operating within the model serving data environment.

16. The method of claim 1, wherein the first dataset and the second dataset each include one or more data elements associated with a shared individual, wherein each data element comprises a linking key that links a data element of the first dataset with a data element of the second dataset.

17. The method of claim 16, further comprising generating a linking database comprising the first dataset, the second dataset, and corresponding linking keys, wherein each linking key is associated with a particular individual.

18. The method of claim 1, further comprising selecting a model training strategy from a strategy library database and training the machine learning model according to the selected model training strategy, wherein the strategy library database comprises a plurality of model training strategies.

19. A system comprising:
one or more computers; and
one or more computer-readable media storing instructions that are operable, when executed by the one or more computers, to perform operations for generating a trained machine learning model trained on a plurality of segregated data sources, the operations comprising:
generating, in a first segregated environment, a first dataset by transforming a first source dataset, the transforming including generating an embedded representation of the first source dataset and adding privacy parameters to the first source dataset;
generating, in a second segregated environment, a second dataset by transforming a second source dataset, the transforming including generating an embedded representation of the second source dataset and adding privacy parameters to the second source dataset;
generating a combined dataset comprising the first dataset and a ground truth dataset from the first segregated data environment combined with the second dataset from the second segregated data environment, wherein the first dataset and the ground truth dataset are stored in a first bridge database, and the second dataset is stored in a second bridge database; and
training a machine learning model with training data, the training data comprising a subset of the combined dataset, wherein model parameters of the trained machine learning model are stored in a storage device.

20. One or more non-transitory computer readable media storing instructions that, when executed by at least one processor, cause the at least one processor to generate a trained machine learning model trained on a plurality of segregated data sources by performing operations comprising:
generating, in a first segregated environment, a first dataset by transforming a first source dataset, the transforming including generating an embedded representation of the first source dataset and adding privacy parameters to the first source dataset;
generating, in a second segregated environment, a second dataset by transforming a second source dataset, the transforming including generating an embedded representation of the second source dataset and adding privacy parameters to the second source dataset;
generating a combined dataset comprising the first dataset and a ground truth dataset from the first segregated data environment combined with the second dataset from the second segregated data environment, wherein the first dataset and the ground truth dataset are stored in a first bridge database, and the second dataset is stored in a second bridge database; and
training a machine learning model with training data, the training data comprising a subset of the combined dataset, wherein model parameters of the trained machine learning model are stored in a storage device.
